(19) United States
(12) Patent Application Publication
(10) Pub. No.: US 2025/0250621 A1
(43) Pub. Date: Aug. 7, 2025

(54) METHODS, COMPOSITIONS, AND KITS FOR DETERMINING THE LOCATION OF AN ANALYTE IN A BIOLOGICAL SAMPLE

(71) Applicant: 10x Genomics, Inc., Pleasanton, CA (US)

(72) Inventors: Christina Galonska, Stockholm (SE); Ariel Royall, Castro Valley, CA (US); Malte Kuhnemund, Stockholm (SE)

(21) Appl. No.: 19/080,120

(22) Filed: Mar. 14, 2025

Related U.S. Application Data
(63) Continuation of application No. 18/649,255, filed on Apr. 29, 2024, now Pat. No. 12,275,988.
Application No. 18/649,255 is a continuation of application No. PCT/US2022/079628, filed on Nov. 10, 2022.
(60) Provisional application No. 63/330,060, filed on Apr. 12, 2022.
(60) Provisional application No. 63/277,817, filed on Nov. 10, 2021.

(51) Int. Cl. C12Q 1/6841; G01N 1/30

(52) U.S. Cl. CPC C12Q 1/6841; G01N 1/30

(57) ABSTRACT

Provided herein are methods, compositions, and kits for the spatial analysis of target nucleic acids, or complements thereof, by their 5′ end.

### CROSS-REFERENCE TO RELATED APPLICATIONS

This application is a continuation of U.S. application Ser. No. 18/649,255, filed on Apr. 29, 2024, which is a continuation of International Application PCT/US2022/079628, with an international filing date of Nov. 10, 2022, which claims the benefit of U.S. Provisional Patent Application No. 63/277,817, filed on Nov. 10, 2021, and U.S. Provisional Patent Application No. 63/330,060, filed on Apr. 12, 2022. The contents of each of these applications is incorporated herein by reference in their entireties.

### SEQUENCE LISTING

This application contains a Sequence Listing that has been submitted electronically as an XML file named 47706-0321002_SL_ST26.xml. The XML file, created on Mar. 14, 2025, is 3,698 bytes in size. The material in the XML file is hereby incorporated by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Sequencing nucleic acid libraries generated from spatial arrays generally biases capture to the 3′ end of the captured analytes. However, 5′ end proximal sequences can contain valuable information about the target analyte. Thus, strategies are needed to sequence regions more than about 1 kilobase away from the 3′ end of analytes, such as 5′ends, in nucleic acid libraries generated from spatial array analysis.

### SUMMARY

The present disclosure features methods of capturing analytes on a spatial array, where the spatial array includes a plurality of capture probes and a capture probe of the plurality of capture probes includes a spatial barcode and a capture domain. Since capture on a spatial array is generally biased for the capture of 3′ ends of nucleic acid analytes, methods are needed to capture the 5′ ends of the nucleic acid analytes, or complements thereof. For example, target RNA nucleic acid analytes can be reverse transcribed with a first primer including a sequence complementary to a target nucleic acid and a functional domain (e.g., a sequencing adapter) to generate an RNA/DNA (e.g., cDNA duplex). The reverse transcriptase can incorporate a non-templated polynucleotide sequence to the 3′ end of the cDNA and a second primer (e.g., a template switching oligonucleotide) can include a sequence complementary to the polynucleotide sequence and a capture sequence. The cDNA can be further extended to incorporate a complement of the second primer (including the capture sequence) at its 3′ end. The target nucleic acid and/or second primer can also be separated from the cDNA (e.g., via enzymatic digestion or heat) resulting in a single-stranded cDNA product that can hybridize to and be captured by a capture probe on the array. Thus, sequences proximal to the 3′ end of the cDNA (corresponding to the 5′ end of the target RNA nucleic acid analyte) are brought proximal to the capture domain and/or barcode of the capture probe.

Provided herein are methods for determining a location of a target nucleic acid in a biological sample, the method including: (a) contacting the biological sample with a first primer including a nucleic acid sequence that is substantially complementary to a sequence in the target nucleic acid and a functional domain; (b) hybridizing the first primer to the target nucleic acid and extending the first primer using the target nucleic acid as a template to generate an extension product; (c) incorporating a polynucleotide sequence including at least three nucleotides to the 3′ end of the extension product; (d) hybridizing a second primer to the polynucleotide sequence including at least three nucleotides of the extension product of (c), where the second primer includes a capture sequence; (e) extending the extension product using the second primer as a template, thereby incorporating a complement of the capture sequence into the extension product; (f) hybridizing the complement of the capture sequence of the extension product in step (e) to a capture domain on an array, where the array includes a plurality of capture probes, and where a capture probe of the plurality of capture probes includes a spatial barcode and the capture domain; and (g) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

In some embodiments, the biological sample is disposed on the array including the plurality of capture probes. In some embodiments, the biological sample is disposed on a first substrate, and where the array including the plurality of capture probes is disposed on a second substrate. In some embodiments, method includes aligning the first substrate with the second substrate, such that at least a portion of the biological sample is aligned with at least a portion of the second substrate.

In some embodiments, the hybridizing in step (f) includes passive migration. In some embodiments, the hybridizing in step (f) includes active migration, and optionally, where the active migration includes electrophoresis.

In some embodiments, the polynucleotide sequence in step (c) includes a homopolynucleotide sequence. In some embodiments, the polynucleotide sequence in step (c) includes a heteropolynucleotide sequence.

In some embodiments, the capture domain includes a fixed sequence.

In some embodiments, the first primer includes a random sequence. In some embodiments, the random sequence includes a random hexamer. In some embodiments, the random sequence includes a random decamer. In some embodiments, the first primer includes a homopolymer sequence. In some embodiments, the homopolymer sequence includes a poly(T) sequence.

In some embodiments, the first primer includes a sequence substantially complementary to a sequence in the target nucleic acid encoding a constant region of an immune cell receptor. In some embodiments, the first primer includes a sequence substantially complementary to a sequence in the target nucleic acid encoding a constant region of a B cell receptor. In some embodiments, the first primer includes a sequence substantially complementary to a sequence in the target nucleic acid encoding a constant region of a T cell receptor.

In some embodiments, the array includes one or more features. In some embodiments, the functional domain includes a primer binding site or a sequencing specific site.

In some embodiments, the target nucleic acid is an RNA. In some embodiments, the RNA is mRNA. In some embodiments, the mRNA includes a sequence encoding a T cell receptor or a fragment thereof. In some embodiments, the mRNA includes a sequence encoding a B cell receptor or a fragment thereof.

In some embodiments, incorporating the polynucleotide sequence to the 3′ end of the extension product in step (c) includes the use of a terminal deoxynucleotidyl transferase. In some embodiments, incorporating the polynucleotide sequence to the 3′ end of the extension product in step (c) includes the use of a reverse transcriptase.

In some embodiments, the second primer includes RNA.

In some embodiments, the method includes removing the target nucleic acid, or any other nucleic acid hybridized to the extension product, before the complement of the capture sequence of the extension product hybridizes to the capture domain of the capture probe on the array. In some embodiments, the removing includes the use of an RNase. In some embodiments, the RNase is RNaseH. In some embodiments, the removing includes use of heat.

In some embodiments, the method includes fixing the biological sample. In some embodiments, fixing the biological sample includes the use of a fixative selected from the group consisting of: ethanol, methanol, acetone, formaldehyde, paraformaldehyde-Triton, glutaraldehyde, and combinations thereof.

In some embodiments, the method includes staining the biological sample. In some embodiments, the staining includes use of eosin and/or hematoxylin. In some embodiments, the staining includes the use of a detectable label selected from the group consisting of a radioisotope, a fluorophore, a chemiluminescent compound, a bioluminescent compound, or a combination thereof.

In some embodiments, the method includes imaging the biological sample.

In some embodiments, the method includes a step of extending the 3′ end of the extension product of step (e) using the capture probe as a template, thereby generating an extended capture product, and/or extending the capture probe using the extension product of step (e) as a template.

In some embodiments, the extended capture product is removed from the capture probe on the array. In some embodiments, the removing includes use of heat.

In some embodiments, the method includes generating a sequence library. In some embodiments, the determining in step (g) includes sequencing. In some embodiments, the sequencing includes high-throughput sequencing.

In some embodiments, the method includes a step of permeabilizing the biological sample, where the permeabilizing includes the use of an organic solvent, a detergent, an enzyme, or a combination thereof. In some embodiments, the permeabilizing includes the use of an endopeptidase, optionally where the endopeptidase is pepsin or proteinase K, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof. In some embodiments, the target nucleic acid encodes V and J sequences of an immune cell receptor. In some embodiments, the target nucleic acid encodes V, D, and J sequences of an immune cell receptor.

In some embodiments, the capture probe includes one or more functional domains, a unique molecular identifier, a cleavage domain, or combinations thereof.

In some embodiments, the biological sample is a tissue sample, optionally where tissue sample is a tissue microarray. In some embodiments, the tissue sample is a fixed tissue sample. In some embodiments, the fixed tissue sample is a formalin-fixed paraffin-embedded tissue sample, a paraformaldehyde-fixed tissue sample, a methanol-fixed tissue sample, or an acetone-fixed tissue sample. In some embodiments, the tissue sample is a fresh frozen tissue sample. In some embodiments, the biological sample is a tissue section. In some embodiments, the tissue section is a fixed tissue section. In some embodiments, the fixed tissue section is a formalin-fixed paraffin-embedded tissue section, a paraformaldehyde-fixed tissue section, a methanol-fixed tissue section, or an acetone-fixed tissue section.

In some embodiments, step (b) includes generating one or more extension products using a plurality of primers. In some embodiments, a primer of the plurality of primers includes a nucleic acid sequence that is substantially complementary to a sequence in the target nucleic acid and a functional domain, where the first primer is included in the plurality of primers; (a) hybridizing the plurality of primers to the target nucleic acid and extending one or more primers from the plurality of primers using the target nucleic acid as a template to generate the one or more extension products; (b) attaching a polynucleotide sequence to the 3′ end of the one or more extension products; (c) hybridizing the second primer to the polynucleotide sequence of the one or more extension products of (b), where the second primer includes a capture sequence; (d) extending the one or more extension products using the second primer as a template, thereby incorporating a complement of the capture sequence into the one or more extension products; (e) hybridizing the complement of the capture sequence of the one or more extension products to a capture domain on an array, where the array includes a plurality of capture probes, and where the capture probe of the plurality of capture probes includes a spatial barcode and the capture domain; and (f) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

In some embodiments, the extending in step (a) includes the use of a reverse transcriptase.

In some embodiments, the plurality of primers hybridize to different sequences in the target nucleic acid. In some embodiments, two or more primers of the plurality of primers hybridize to different sequences in the target nucleic acid. In some embodiments, ten or more primers of the plurality of primers hybridize to different sequences in the target nucleic acid. In some embodiments, fifty or more primers of the plurality of primers hybridize to different sequences in the target nucleic acid. In some embodiments, one hundred or more primers of the plurality of primers hybridize to different sequences in the target nucleic acid.

In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid.

In some embodiments, the extending in step (a) includes the use of a reverse transcriptase and a helicase. In some embodiments, the method includes use of one or more single-stranded DNA binding proteins. In some embodiments, the one or more single-stranded DNA binding proteins includes one or more of: Tth RecA, E. coli RecA, T4 gp32 and ET-SSB. In some embodiments, the helicase has strand displacement activity.

In some embodiments, the extending in step (a) includes the use of a superhelicase and a reverse transcriptase. In some embodiments, the superhelicase is selected from the group consisting of: Rep, PrcA, UvrB, RecBCD, and Tte-Uvrd. In some embodiments, the superhelicase has strand displacement activity.

In some embodiments, the method includes generating two or more extension products from the primer of the plurality of primers. In some embodiments, the one or more extension products include different sequence lengths.

Also provided herein are kits including: (a) an array including a plurality of capture probes, where a capture probe of the plurality of capture probes includes: (i) a spatial barcode; and (ii) a capture domain that binds a capture sequence, or a complement thereof; (b) a first primer including a sequence substantially complementary to a target nucleic acid and a functional domain, where the functional domain includes a primer binding sequence or a sequencing specific sequence; (c) a second primer including a capture sequence; and optionally (d) instructions for performing any of the methods described herein.

In some embodiments, the capture probe includes one or more additional functional domains, a unique molecular identifier, a cleavage domain, or combinations thereof.

In some embodiments, the kit includes a reverse transcriptase and a polymerase.

In some embodiments, the kit includes a plurality of primers, where the first primer is included in the plurality of primers.

In some embodiments, the capture domain includes a fixed sequence.

In some embodiments, the second primer includes RNA.

In some embodiments, the kit includes a helicase. In some embodiments, the kit includes one or more single-stranded DNA binding proteins selected from the group consisting of Tth RecA, E. coli RecA, T4 gp32 and ET-SSB.

In some embodiments, the kit includes a superhelicase, where the superhelicase is selected from the group consisting of: Rep, PrcA, UvrB, RecBCD, and Tte-Uvrd.

Also provided herein are methods for determining locations of target nucleic acids in a biological sample, the method including: (a) contacting the biological sample with a plurality of primers, where the plurality of primers includes nucleic acid sequences that hybridize to complementary sequences in the target nucleic acids and a functional domain; (b) hybridizing the plurality of primers to the target nucleic acids and extending one or more of the plurality of primers using the target nucleic acids as a template to generate one or more extension products; (c) incorporating a polynucleotide sequence including at least three nucleotides to the 3′ end of the one or more extension products; (d) hybridizing a second primer to the polynucleotide sequence including the at least three nucleotides of the one or more extension products of (c), where the second primer includes a capture sequence; (e) extending the one or more extension products using the second primer as a template, thereby incorporating a complement of the capture sequence into the one or more extension products; (f) hybridizing the complement of the capture sequence of the one or more extension products to capture domains on an array, where the array includes a plurality of capture probes, and where the plurality of capture probes collectively includes spatial barcodes and the capture domains; and (g) determining (i) the sequences of the spatial barcodes, or a complement thereof, and (ii) all or a portion of the sequence of the target nucleic acids, or a complement thereof, and using the determined sequences of (i) and (ii) to determine the locations of the target nucleic acids in the biological sample.

In some embodiments, the biological sample is disposed on the array including the plurality of capture probes. In some embodiments, the biological sample is disposed on a first substrate, and where the array including the plurality of capture probes is disposed on a second substrate. In some embodiments, includes aligning the first substrate with the second substrate, such that at least a portion of the biological sample is aligned with at least a portion of the array.

In some embodiments, the hybridizing in step (f) includes passive migration. In some embodiments, the hybridizing in step (f) includes active migration, and optionally, where the active migration includes electrophoresis.

In some embodiments, the polynucleotide sequence in step (c) includes a homopolynucleotide sequence. In some embodiments, the polynucleotide sequence in step (c) includes a heteropolynucleotide sequence.

In some embodiments, the capture domain includes a fixed sequence.

In some embodiments, a primer in the plurality of primers includes a random sequence. In some embodiments, the random sequence includes a random hexamer. In some embodiments, the random sequence includes a random decamer. In some embodiments, the plurality of primers includes a homopolymer sequence. In some embodiments, the homopolymer sequence includes a poly(T) sequence.

In some embodiments, the plurality of primers includes one or more sequences substantially complementary to one or more sequences in the target nucleic acids encoding a constant region of an immune cell receptor. In some embodiments, the plurality of primers includes one or more sequences substantially complementary to one or more sequences in the target nucleic acids encoding a constant region of a B cell receptor, preferably an antibody. In some embodiments, the plurality of primers includes one or more sequences substantially complementary to one or more sequences in the target nucleic acids encoding a constant region of a T cell receptor.

In some embodiments, the array includes one or more features.

In some embodiments, the functional domain includes a primer binding site or a sequencing specific site.

In some embodiments, the target nucleic acids are RNAs. In some embodiments, the RNAs are mRNAs. In some embodiments, the mRNAs include a sequence encoding a T cell receptor or a fragment thereof. In some embodiments, the mRNAs include a sequence encoding a B cell receptor or a fragment thereof.

In some embodiments, the extending in step (b) includes the use of a reverse transcriptase.

In some embodiments, the plurality of primers hybridize to different sequences in the target nucleic acids. In some embodiments, two or more primers of the plurality of primers hybridize to different sequences in the target nucleic acids. In some embodiments, ten or more primers of the plurality of primers hybridize to different sequences in the target nucleic acids. In some embodiments, fifty or more primers of the plurality of primers hybridize to different sequences in the target nucleic acids. In some embodiments, one hundred or more primers of the plurality of primers hybridize to different sequences in the target nucleic acids.

In some embodiments, the reverse transcriptase has strand displacement activity. In some embodiments, the strand displacement activity of the reverse transcriptase displaces one or more primers of the plurality of primers from the target nucleic acids. In some embodiments, the strand displacement activity of the reverse transcriptase displaces the one or more extension products from the target nucleic acids.

In some embodiments, the extending in step (b) includes the use of a reverse transcriptase and a helicase. In some embodiments, the method includes the use of one or more single-stranded DNA binding proteins. In some embodiments, the one or more single-stranded DNA binding proteins includes one or more of: Tth RecA, E. coli RecA, T4 gp32 and ET-SSB. In some embodiments, the helicase has strand displacement activity.

In some embodiments, the extending in step (b) includes the use of a superhelicase and a reverse transcriptase. In some embodiments, the superhelicase is selected from the group consisting of: Rep, PrcA, UvrB, RecBCD, and Tte-Uvrd. In some embodiments, the superhelicase has strand displacement activity.

In some embodiments, the method includes generating two or more extension products from a primer of the plurality of primers. In some embodiments, the one or more extension products include different sequence lengths.

In some embodiments, incorporating the polynucleotide sequence to the 3′ end of the one or more extension products in step (c) includes the use of a terminal deoxynucleotidyl transferase or a reverse transcriptase.

In some embodiments, the second primer includes RNA.

In some embodiments, the functional sequence is a primer binding sequence or a sequencing specific sequence.

In some embodiments, the method includes removing the target nucleic acids, or any other nucleic acid hybridized to the one or more extension products, before the complement of the capture sequence of the one or more extension products hybridizes to the plurality of capture domains on the array. In some embodiments, the removing includes the use of an RNase. In some embodiments, the RNase is RNaseH. In some embodiments, the removing includes use of heat.

In some embodiments, the method includes fixing the biological sample. In some embodiments, fixing the biological sample includes the use of a fixative selected from the group consisting of: ethanol, methanol, acetone, formaldehyde, paraformaldehyde-Triton, glutaraldehyde, and combinations thereof.

In some embodiments, the method includes staining the biological sample. In some embodiments, the staining includes use of eosin and/or hematoxylin. In some embodiments, the staining includes the use of a detectable label selected from the group consisting of a radioisotope, a fluorophore, a chemiluminescent compound, a bioluminescent compound, or a combination thereof.

In some embodiments, the method includes imaging the biological sample.

In some embodiments, the method includes a step of extending the 3′ end of the one or more extension products of step (e) using the capture probes as a template, thereby generating one or more extended capture products, and/or extending the capture probes using the one or more extension product of step (e) as a template.

In some embodiments, the one or more extended capture products is removed from the capture probes on the array. In some embodiments, the removing includes use of heat.

In some embodiments, the method includes generating a sequence library. In some embodiments, the determining in step (g) includes sequencing. In some embodiments, the sequencing includes high-throughput sequencing.

In some embodiments, the method includes a step of permeabilizing the biological sample, where the permeabilizing includes the use of an organic solvent, a detergent, an enzyme, or a combination thereof. In some embodiments, the permeabilizing includes the use of an endopeptidase, optionally where the endopeptidase is pepsin or proteinase K, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

In some embodiments, the target nucleic acids encode V and J sequences of an immune cell receptor. In some embodiments, the target nucleic acids encode V, D, and J sequences of an immune cell receptor.

In some embodiments, a capture probe in the plurality of capture probes includes one or more functional domains, a unique molecular identifier, a cleavage domain, or combinations thereof.

In some embodiments, the biological sample is a tissue sample, optionally where tissue sample is a tissue microarray. In some embodiments, the tissue sample is a fixed tissue sample. In some embodiments, the fixed tissue sample is a formalin-fixed paraffin-embedded tissue sample, a paraformaldehyde-fixed tissue sample, a methanol-fixed tissue sample, or an acetone-fixed tissue sample. In some embodiments, the tissue sample is a fresh frozen tissue sample. In some embodiments, the biological sample is a tissue section. In some embodiments, the biological sample is a fresh-frozen tissue section. In some embodiments, the tissue section is a fixed tissue section. In some embodiments, the fixed tissue section is a formalin-fixed paraffin-embedded tissue section, a paraformaldehyde-fixed tissue section, a methanol-fixed tissue section, or an acetone-fixed tissue section.

Also provided herein are composition including: a) a target nucleic acid; and b) one or more extension products hybridized to the target nucleic acid, where the one or more extension products includes in a 5′ to 3′ direction: i) a primer hybridized to a region of the target nucleic acid that encodes a constant region of a B cell receptor or a T cell receptor, where the primer includes a functional domain; ii) a complementary sequence of a region of the target nucleic acid that encodes V and J sequences of the B cell receptor or the T cell receptor; and iii) a polynucleotide sequence.

In some embodiments, the composition includes a second primer hybridized to the polynucleotide sequence, where the second primer includes a capture sequence. In some embodiments, the one or more extension products includes a complement of the capture sequence.

In some embodiments, the target nucleic acid is RNA. In some embodiments, the RNA is mRNA.

In some embodiments, the functional domain includes a primer binding site. In some embodiments, the functional domain includes a sequencing specific site.

In some embodiments, the second primer includes RNA.

In some embodiments, the composition includes a reverse transcriptase. In some embodiments, the composition includes a helicase. In some embodiments, the composition includes one or more single-stranded DNA binding proteins selected from the group consisting of: Tth RecA, E. coli RecA, T4 gp32 and ET-SSB. In some embodiments, the composition includes one or more superhelicases selected from the group consisting of: Rep, PrcA, UvrB, RecBCD, and Tte-Uvrd.

In some embodiments, the polynucleotide sequence includes a homopolynucleotide sequence. In some embodiments, the polynucleotide sequence includes a heteropolynucleotide sequence.

In some embodiments, the complementary sequence of the region of the target nucleic acid encodes a D sequence of the B cell receptor or the T cell receptor.

Also provided herein are compositions including: one or more extension products, where the one or more extension products include in a 5′ to 3′ direction: i) a primer, where the primer includes a functional domain; ii) a sequence substantially complementary to a region of a target nucleic acid that encodes V and J sequences of an immune cell receptor; and iii) a polynucleotide sequence, where the one or more extension products is hybridized to a capture domain on an array, where the array includes a plurality of capture probes, and where a capture probe of the plurality of capture probes includes a spatial barcode and the capture domain.

In some embodiments, the polynucleotide sequence includes a heteropolynucleotide sequence. In some embodiments, the polynucleotide sequence includes a homopolynucleotide sequence.

In some embodiments, the capture probe includes a cleavage domain, one or more functional domains, a unique molecular identifier, or a combination thereof.

In some embodiments, the functional domain includes a primer binding sequence or a sequencing specific sequence.

In some embodiments, the sequence substantially complementary to a region of the target nucleic acid encodes a D sequence of the immune cell receptor. In some embodiments, the polynucleotide sequence is not complementary to i) the target nucleic acid that encodes a constant region of a B cell receptor or a T cell receptor, or ii) the target nucleic acid that encodes V and J sequences of an immune cell receptor.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram showing reverse transcription of a target nucleic acid with a first primer and the addition of the complement of a capture sequence into an extension product which is capable of hybridizing to a capture domain of a capture probe.

FIG. 3 is a schematic diagram showing capture and extension on an array of the extension product (e.g., cDNA product) shown in FIG. 2 and extension of the capture probe and the captured extension product (e.g., cDNA product) followed by release of the extended capture product.

FIG. 4 is a schematic diagram showing reverse transcription of a target nucleic acid with a plurality of primers where the reverse transcription occurs with a reverse transcriptase with strand displacement activity or a reverse transcriptase with a helicase or a superhelicase (top). In the embodiment shown, the primers hybridize to a region of the target nucleic acid that encodes for a constant region of an immune cell receptor and generate one or more extension products of varying lengths that include V(D)J sequences depending on where the primers hybridize to the target nucleic acid that encodes for a constant region of an immune cell receptor (bottom).

FIGS. 5A-B are mouse brain images showing fluorescently labeled cDNA post reverse transcription (FIG. 5A) and post permeabilization and cDNA extension (FIG. 5B).

FIGS. 6A-B show mouse brain images. FIG. 6A shows a brightfield image and FIG. 6B shows fluorescently labeled extended cDNA generated by extension in the presence of Cy3-dCTPs.

FIGS. 7A-C shows spatial gene expression clusters (FIG. 7A), the corresponding t-SNE plot (FIG. 7B), and spatial gene expression heat map (FIG. 7C).

FIGS. 8A-D show spatial gene expression clustering with a first primer including a poly(T) sequence (FIG. 8A) and the corresponding t-SNE plot (FIG. 8B) and spatial gene expression clustering with a first primer including a random decamer (FIG. 8C) and the corresponding t-SNE plot (FIG. 8D).

FIG. 9 shows fluorescently labeled extended cDNA post permeabilization and cDNA extension in mouse brain tissue using a template switch ribonucleotide with an alternative handle.

FIGS. 10A-B are graphs showing correlation between fresh frozen capture using standard Visium spatial gene expression (10× Genomics) and spatial 5′ end capture (FIG. 10A) and a graph showing normalized position of each mapped read within the full-length transcript and confirming successful 5′ enrichment with a primer including a random decamer (FIG. 10B).

FIG. 11 is a schematic diagram depicting an exemplary sandwiching process between a first substrate comprising a biological sample and a second substrate comprising a spatially barcoded array.

FIGS. 12A-B are schematic diagrams depicting exemplary sandwiching embodiments. FIG. 12A shows an exemplary sandwiching process where a first substrate including a biological sample and a second substrate are brought into proximity with one another and a liquid reagent drop is introduced on the second substrate in proximity to the capture probes. FIG. 12B shows a fully formed sandwich configuration creating a chamber formed from one or more spacers, the first substrate, and the second substrate including spatially barcoded capture probes.

### DETAILED DESCRIPTION

The present disclosure features methods of capturing analytes on a spatial array, where the spatial array includes a plurality of capture probes and a capture probe of the plurality of capture probes includes a spatial barcode and a capture domain. Sequences more than about 1 kilobase away from the 3′ end of nucleic acid analytes are generally not captured in nucleic acid sequencing libraries, however, some analytes, such as nucleic acid analytes encoding immune cell receptors (e.g., B cell receptor, T cell receptor) contain sequences of interest (e.g., VDJ sequences) more than 1 kilobase away from the 3′ end of the analyte. Since capture on a spatial array generally biases the 3′ end of nucleic acid analytes (e.g., mRNA), methods are needed to capture the 5′ end of the nucleic acid analyte, or a complement thereof (e.g., a proxy of the analyte).

Analytes, such as mRNA, can be reverse transcribed by hybridizing to the target mRNA a first primer that includes a sequence complementary to the target mRNA and a functional domain (e.g., a sequence domain for use in sequencing, a primer binding domain, etc.) to generate an RNA/DNA (e.g., RNA/cDNA) duplex. A polynucleotide sequence can be incorporated at the end of the cDNA. For example, a reverse transcriptase or a terminal transferase can add a polynucleotide sequence in a template-independent manner (e.g., at least three non-templated nucleotides). In some embodiments, the polynucleotide sequence is a heteropolynucleotide sequence (e.g., CGC). In some embodiments, the polynucleotide sequence is a homopolynucleotide sequence (e.g., CCC). A second primer that includes a complement of the polynucleotide sequence and a capture sequence can be hybridized to the polynucleotide sequence incorporated at the end (e.g., the 3′ end) of the cDNA. The 3′ end of the cDNA is extended using the second primer as a template (resulting in an RNA/DNA (e.g., RNA/cDNA) duplex). The target RNA can be removed (e.g., via digestion, denaturation, etc.) resulting in a single-stranded product that serves as a proxy of the target analyte which can be captured by a capture probe on a spatial array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein. Examples of nucleic acid analytes include, but are not limited to, DNA (e.g., genomic DNA, cDNA) and RNA, including coding and non-coding RNA (e.g., mRNA, rRNA, tRNA, ncRNA).

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

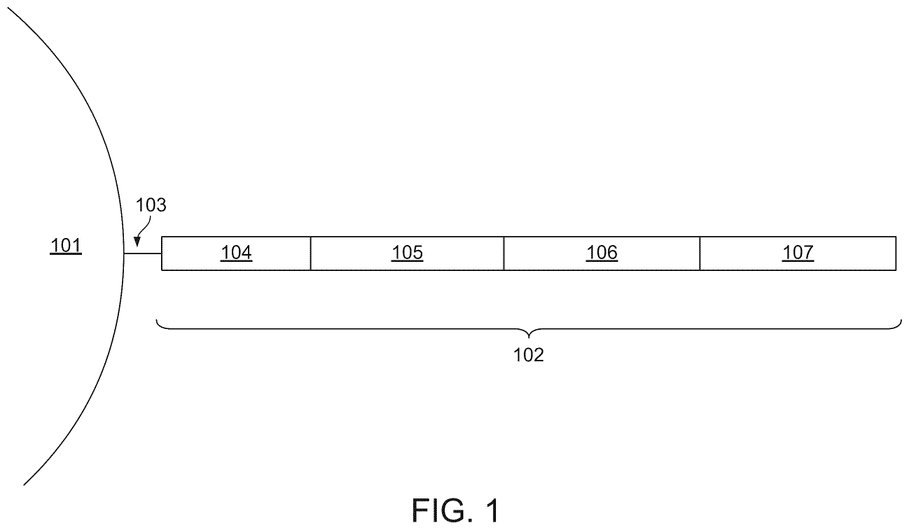

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature (e.g., a bead, a well, a spot on an array). In some embodiments, the UMI sequence 106 of a capture probe attached to a given feature is different from the UMI sequence of a different capture probe attached to the given feature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., an extension product, a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding or incorporating to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

As used herein, “an extension product” refers to an analyte, such as RNA (e.g., mRNA), that has been reverse transcribed (e.g., with a reverse transcriptase) to generate cDNA. In some embodiments, the cDNA is hybridized to the RNA (e.g., RNA/cDNA duplex). In some embodiments, the cDNA is hybridized to the mRNA. In some embodiments, the extension product is further extended by adding or incorporating a polynucleotide sequence (e.g., a heteropolynucleotide sequence, a homopolynucleotide sequence) to the 3′ end of the extension product (e.g., cDNA). For example, a reverse transcriptase or a terminal transferase can add at least three nucleotides (e.g., a polynucleotide sequence) to the 3′ end of the extension product in a template-independent manner. In some embodiments, the extension product is further extended when a second primer hybridizes to the polynucleotide sequence and the extension product is further extended using the second primer as a template. In some embodiments, the second primer includes a capture sequence which is incorporated (e.g., a complement thereof) into the sequence of the extension product. In some embodiments, the RNA (e.g., mRNA) is removed (e.g., by digestion) from the extension product. In such embodiments, the term extension product also refers to a single-stranded DNA (e.g., cDNA) product that includes a complement of the target nucleic acid (e.g., RNA, mRNA).

As used herein, “an extended capture product” refers to an extension product that has been captured on a spatial array and extended using the capture probe as a template. For example, an extension product, as described herein, after capture by a capture probe can be extended to include a capture sequence, or a complement thereof, that is capable of hybridizing to a capture domain of a capture probe. In some embodiments, when the extension product hybridizes to the capture domain of the capture probe, an end of the extension product (e.g., a 3′ end) can be extended to generate the extended capture product. In such examples, the extended capture product includes the domains (e.g., a UMI, a spatial barcode, one or more functional domains, etc.) present in the capture probe on the spatial array. In some embodiments, the extended capture product is released from the capture probe and collected for downstream applications, such as amplification and sequencing.

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe such as an extended capture product), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof), such as an extension product, can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### 5′ Capture of Target Nucleic Acids

The present disclosure features methods of capturing analytes on a spatial array, where the spatial array includes a plurality of capture probes and a capture probe of the plurality of capture probes includes a spatial barcode and a capture domain. In some embodiments, the analyte is a target nucleic acid. Since capture on a spatial array can be biased towards 3′ end capture of nucleic acid analytes, methods are needed to determine the sequence of the 5′ end of nucleic acid analytes (e.g., by capturing the 5′ end of the nucleic acid analyte), or a complement thereof (e.g., a proxy of the analyte). For example, target nucleic acid analytes (e.g., RNA) can be reverse transcribed with a first primer including a sequence substantially complementary to the target nucleic acid and a functional domain, such as a primer binding site or a sequencing specific site to generate an RNA/DNA (e.g., cDNA) duplex. An enzyme, such as a reverse transcriptase or terminal transferase, can add non-templated nucleotides to the 3′ end of the cDNA. For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA. A second primer that includes a sequence substantially complementary to the non-templated nucleotides (e.g., the polynucleotide sequence) and a capture sequence can hybridize to the non-templated nucleotides (e.g., the polynucleotide sequence) added to the end of the cDNA. In some embodiments, the second primer includes an RNA sequence (e.g., one or more ribonucleotides). The cDNA is extended using the second primer as a template thereby incorporating the complement of the capture sequence into the cDNA. The complement of the capture sequence can hybridize to the capture domain of the capture probe on the substrate. The target nucleic acid and/or the ribo-second primer can be removed (e.g., digested, denatured, etc.) resulting in a single-stranded DNA product. The single-stranded DNA product can include the functional domain at its 5′ end, a copy of the target analyte (e.g., cDNA), and a complement of the capture sequence that is capable of binding (e.g., hybridizing) to a capture domain of a capture probe on the array at its 3′ end.

Target nucleic acids can include a nucleic acid molecule with a nucleic acid sequence encoding at least a portion of a V-J sequence or a V(D)J sequence of an immune cell receptor (e.g., a T cell receptor or a fragment thereof or a B cell receptor or a fragment thereof). Target nucleic acids can include a nucleic acid molecule with a nucleic acid sequence encoding an antibody. In some embodiments, the target nucleic acid is RNA. In some embodiments, the RNA is mRNA. In some embodiments, the target nucleic acids are nucleic acids encoding immune cell receptors. In some embodiments, target nucleic acids encoding immune cell receptors identify clonotype populations from a biological sample. In some embodiments, target nucleic acids include a constant region, such as a sequence encoding a constant region of an immune cell receptor (e.g., antibody). In some embodiments, target nucleic acids include a variable region, such as a sequence encoding a variable region of an immune cell receptor (e.g., antibody).

In some embodiments, the target nucleic acid encodes an immune cell receptor. In some embodiments, the immune cell receptor is a B cell receptor. In some embodiments, the B cell receptor includes an immunoglobulin kappa light chain. In some embodiments, the target nucleic acid includes a sequence encoding a CDR3 region of the immunoglobulin kappa light chain. In some embodiments, the target nucleic acid includes a sequence encoding one or both of CDR1 and CDR2 of the immunoglobulin kappa light chain. In some embodiments, the target nucleic acid includes a sequence encoding a full-length variable domain of the immunoglobulin kappa light chain.

In some embodiments, the B cell receptor includes an immunoglobulin lambda light chain. In some embodiments, the target nucleic acid includes a sequence encoding a CDR3 of the immunoglobulin lambda light chain. In some embodiments, the target nucleic acid includes a sequence encoding one or both of CDR1 and CDR2 of the immunoglobulin lambda light chain. In some embodiments, the target nucleic acid includes a sequence encoding a full-length variable domain of the immunoglobulin lambda light chain.

In some embodiments, the B cell receptor includes an immunoglobulin heavy chain. In some embodiments, the target nucleic acid includes a sequence encoding a CDR3 of the immunoglobulin heavy chain. In some embodiments, the target nucleic acid includes a sequence encoding one or both of CDR1 and CDR2 of the immunoglobulin heavy chain. In some embodiments, the target nucleic acid includes a sequence encoding a full-length variable domain of the immunoglobulin heavy chain.

In some embodiments, the immune cell receptor is a T cell receptor. In some embodiments, the T cell receptor includes a T cell receptor alpha chain. In some embodiments, the target nucleic acid includes a sequence encoding a CDR3 of the T cell receptor alpha chain. In some embodiments, the target nucleic acid includes a sequence encoding one or both of CDR1 and CDR2 of the T cell receptor alpha chain. In some embodiments, the target nucleic acid includes a sequence encoding a full-length variable domain of the T cell receptor alpha chain.

In some embodiments, the T cell receptor includes a T cell receptor beta chain. In some embodiments, the target nucleic acid includes a sequence encoding a CDR3 of the T cell receptor beta chain. In some embodiments, the target nucleic acid includes a sequence encoding one or both of CDR1 and CDR2 of the T cell receptor beta chain. In some embodiments, the target nucleic acid further includes a full-length variable domain of the T cell receptor beta chain.

While useful for analysis of immune cell receptor analytes, such as nucleic acids encoding immune cell receptors, the disclosed methods are not limited thereto. Thus, the disclosed methods can be useful for analysis of nucleotide sequence at and/or proximal to the 5′ end of any target nucleic acid, such as, genomic DNA, lncRNA, and the like.

In some embodiments, the methods included herein include determining the complementarity determining regions (CDRs) of a T cell receptor or a fragment thereof, and/or a B cell receptor or a fragment thereof (e.g., an antibody or fragment thereof).

Thus, provided herein are methods for determining a location of a target nucleic acid in a biological sample, the method including: (a) contacting the biological sample with a first primer including a nucleic acid sequence that is substantially complementary to a sequence in the target nucleic acid and a functional domain; (b) hybridizing the first primer to the target nucleic acid and extending the first primer using the target nucleic acid as a template to generate an extension product; (c) incorporating a polynucleotide sequence including at least three nucleotides to the 3′ end of the extension product; (d) hybridizing a second primer to the polynucleotide sequence including at least three nucleotides of the extension product of (c), where the second primer includes a capture sequence; (e) extending the extension product using the second primer as a template, thereby incorporating a complement of the capture sequence into the extension product; (f) hybridizing the complement of the capture sequence of the extension product in step (e) to a capture domain on an array, where the array includes a plurality of capture probes, and where a capture probe of the plurality of capture probes includes a spatial barcode and the capture domain; and (g) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

The methods described herein can also use a plurality of primers, where the first primer is included in the plurality of primers. For example, the plurality of primers can hybridize to a target nucleic acid at different locations in the target nucleic acid (e.g., where the primers are substantially complementary to the target nucleic acid) and subsequently be extended. Extending the plurality of primers generates one or more extension products that include a complement of the capture sequence as described herein. For example, the methods provided herein can include providing a plurality of primers wherein each primer includes a sequence that hybridizes to a substantially complementary sequence in the target nucleic acid and a functional domain, wherein the first primer is comprised in the plurality of primers and (a) hybridizing the plurality of primers to the target nucleic acid and extending one or more primers from the plurality of primers using the target nucleic acid as a template to generate the one or more extension products; (b) attaching a polynucleotide sequence to the 3′ end of the one or more extension products; (c) hybridizing the second primer to the polynucleotide sequence of the one or more extension products of (b), where the second primer includes a capture sequence; (d) extending the one or more extension products using the second primer as a template, thereby incorporating a complement of the capture sequence into the one or more extension products; (e) hybridizing the complement of the capture sequence of the one or more extension products to a capture domain on an array, where the array includes a plurality of capture probes, and where the capture probe of the plurality of capture probes includes a spatial barcode and the capture domain; and (f) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

Also provided herein are methods for determining locations of target nucleic acids in a biological sample, the method including: (a) contacting the biological sample with a plurality of primers, where the plurality of primers includes nucleic acid sequences that hybridize to substantially complementary sequences in the target nucleic acids and a functional domain; (b) hybridizing the plurality of primers to the target nucleic acids and extending one or more of the plurality of primers using the target nucleic acids as a template to generate one or more extension products; (c) incorporating a polynucleotide sequence including at least three nucleotides to the 3′ end of the one or more extension products; (d) hybridizing a second primer to the polynucleotide sequence including the at least three nucleotides of the one or more extension products of (c), where the second primer includes a capture sequence; (e) extending the one or more extension products using the second primer as a template, thereby incorporating a complement of the capture sequence into the one or more extension products; (f) hybridizing the complement of the capture sequence of the one or more extension products to capture domains on an array, where the array includes a plurality of capture probes, and where the plurality of capture probes collectively includes spatial barcodes and the capture domains; and (g) determining (i) the sequences of the spatial barcodes, or a complement thereof, and (ii) all or a portion of the sequence of the target nucleic acids, or a complement thereof, and using the determined sequences of (i) and (ii) to determine the locations of the target nucleic acids in the biological sample.

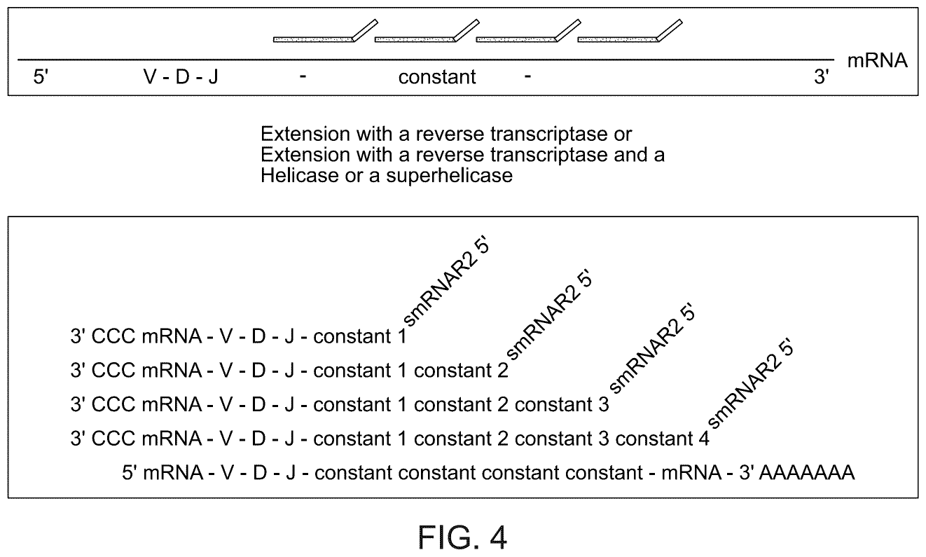

In embodiments where a plurality of primers are used, the plurality of primers can hybridize to different sequences in the target nucleic acid. For example, the plurality of primers can hybridize to adjacent sequences (e.g., “tiling”) on the target nucleic acid. Adjacent sequences can be, but are not necessarily, contiguous. In some embodiments, adjacent sequences can be within 1-10 nucleotides of each other. In some embodiments, the plurality of primers hybridize to non-adjacent sequences on the target nucleic acid. In some embodiments, two or more primers of the plurality of primers hybridize to different sequences in the target nucleic acid. In some embodiments, ten or more primers of the plurality of primers hybridize to different sequences in the target nucleic acid. In some embodiments, fifty or more primers of the plurality of primers hybridize to different sequences in the target nucleic acid. In some embodiments, one hundred or more primers of the plurality of primers hybridize to different sequences in the target nucleic acid. In some embodiments, a plurality of extension products can be generated by extension of the plurality of primers. The resulting extension products can have differing lengths depending on the exact location in the target nucleic acid from which they were primed. Thus, in some embodiments, extension products (e.g., cDNA) of different lengths and/or sequences can be generated from the same target nucleic acid (e.g., mRNA). See, e.g., FIG. 4.

In embodiments where the target nucleic acid includes a sequence that encodes an immune cell receptor, the plurality of primers preferably hybridize to a region of the target nucleic acid that encodes a constant region of the immune cell receptor.

In some embodiments, the extension product(s) that hybridize to the capture domains of the plurality of capture probes can migrate (e.g., diffuse) towards the capture probes through passive migration such as gravity. In some embodiments, the extension product(s) that hybridize to the capture domains of the plurality of capture probes can migrate toward the capture probes through active migration. In some embodiments, the active migration includes electrophoresis.

In some embodiments, the array includes one or more features (e.g., any of the features described herein). In some embodiments, the one or more features includes a bead.

As described herein, a capture probe refers to any molecule capable of capturing (directly or indirectly) and/or labelling an analyte (e.g., an analyte of interest, such as a nucleic acid) in a biological sample. In some embodiments, the capture probe is a nucleic acid. In some embodiments, the capture probe is DNA. In some embodiments, the capture probe includes a barcode (e.g., a spatial barcode) and/or a unique molecular identifier (UMI)) and a capture domain. In some embodiments, a capture probe can include a cleavage domain and/or one or more functional domains (e.g., a primer-binding site or a sequencing specific site, such as for next-generation sequencing (NGS)).

In some embodiments, the capture domain includes a fixed sequence. As used herein, a “fixed sequence” is a non-random sequence. A “fixed sequence” can be substantially complementary to the complement of the capture sequence that is incorporated into the extension product(s) described herein. For example, the capture domain (e.g., the fixed sequence therein) can be substantially identical to the capture sequence comprised in the second primer. In some embodiments, the plurality of capture probes on an array include the same fixed sequence. The methods described herein can use different capture sequences included in the second primer which are incorporated (e.g., a complement thereof) into the extension product(s). For example, it is only necessary for the complement of the capture sequence in the extension products and the capture domain to be substantially complementary to each other, such that the capture domain including a fixed sequence can hybridize to and/or capture the extension product(s).

In some embodiments, the first primer includes a sequence that is substantially complementary to a sequence in a target nucleic acid. The sequence substantially complementary to the target nucleic acid can be a gene specific sequence, which, for example, can allow for selective capture of a desired target nucleic. In some embodiments, the first primer includes a homopolymer sequence and a functional domain. In some embodiments, the homopolymer sequence is a poly(T) sequence. In some embodiments, the first primer includes a random sequence and a functional domain. In some embodiments, the random sequence includes 5, 6, 7, 8, 9, 10, 11, 12, 13, 14, 15, 16, 17, 18, 19, 20, or more random nucleotides. In some embodiments, the random sequence is a random hexamer. In some embodiments, the random sequence is a random decamer. In some embodiments, the functional domain is a primer binding site. In some embodiments, the functional domain is a sequencing specific site.

In some embodiments, the extending in step (a) includes the use of a reverse transcriptase. In some embodiments, the reverse transcriptase has strand displacement activity. In some embodiments, the strand displacement activity of the reverse transcriptase displaces one or more primers of the plurality of primers from the target nucleic acid. In some embodiments, the strand displacement activity of the reverse transcriptase displaces the one or more extension products from the target nucleic acid.

In some embodiments, the extending in step (a) includes the use of a reverse transcriptase and a helicase. As defined herein, “helicases” are enzymes that catalyze the reaction of separating or unwinding the helical structure of nucleic acid complexes, e.g. double-stranded DNA, double-stranded RNA, or DNA:RNA complexes, into single stranded components. Helicases generally are known to use a nucleoside triphosphate (NTP) (e.g., ATP) hydrolysis as a source of energy. In some embodiments, the method includes one or more single-stranded DNA binding proteins. In some embodiments, the one or more single-stranded DNA binding proteins comprises one or more of: Tth RecA, E. coli RecA, T4 gp32 and ET-SSB. As defined herein, “single-stranded DNA binding proteins” or “SSBs” are proteins that bind to single-stranded DNA. SSBs, or functional equivalents, are found in a variety of organisms, including eukaryotes and bacteria. Single-stranded DNA is produced, for example, during aspects of DNA metabolism, DNA replication, DNA recombination, and DNA repair. As well as stabilizing single-stranded DNA, SSB proteins bind to and modulate the function of numerous proteins involved in the aforementioned processes. In addition, SSB proteins can destabilize ends of double-stranded nucleic acid (e.g., dsDNA). In some embodiments, the helicase has strand displacement activity. For example, the helicases and single-stranded DNA binding proteins described herein can unwind DNA:RNA complexes that allow a reverse transcriptase to reverse transcribe the target nucleic acid. In some embodiments, a helicase can unwind a first target nucleic acid: extension product complex (e.g., mRNA:cDNA) that is downstream of a second target nucleic acid: extension product complex, such that the target nucleic acid from the first complex (with or without the help of the SSBs) becomes available as template for further extension of the extension product in the second complex.

In some embodiments, helicases, including superhelicases, are also used in conjunction with SSB proteins during nucleic acid amplification. In some embodiments, the extending in step (a) includes the use of a superhelicase and a reverse transcriptase. In some embodiments, the superhelicase is selected from the group consisting of: Rep, PrcA, UvrB, RecBCD, and Tte-Uvrd. As defined herein a “superhelicase” is a mutant and/or a derivative of a helicase. Superhelicases can have increased processivity compared to helicases due to one or more derivations that can include mutated gene or substituted polypeptide sequences and/or cross-linked protein domains. Additionally, superhelicases can unwind double-stranded nucleic acid complexes without single-stranded DNA binding protein(s). In some embodiments, the helicase is a superhelicase. In some embodiments, superhelicases have increased processivity relative to helicases. In some embodiments, the superhelicase has strand displacement activity. For example, superhelicases can unwind double-stranded nucleic acid complexes longer than 150 base pairs.

In some embodiments, the method includes generating two or more extension products from the primer of the plurality of primers. For example, a single primer can facilitate reverse transcription for more than a single extension reaction resulting in 2, 3, 4, 5, 6, 7, 8, 9, 10, 20, 30, 40, 50, 100, 200, 300, 400, 500, 600, 700, 800, 900, 1000, or more extension products produced from a single primer. In some embodiments, the two or more extension products generated from the same primer include the same sequence and/or length.

In some embodiments, the plurality of primers comprises multiple unique primers, where each unique primer includes a unique sequence and/or length. The plurality of primers can include multiple copies (e.g., 2, 10, 20, 50, 100, 1000, 10000 or more) of each unique primer. In some embodiments, each unique primer in the plurality of primers hybridizes to a distinct sequence within the target nucleic acid. Thus, in some embodiments, the one or more extension products include different sequences and/or lengths. For example, when a plurality of primers are hybridized to a target nucleic acid and extended, the one or more extension products can have varying sequences and/or lengths.

In some embodiments, the extending in step (b) includes the use of a reverse transcriptase (e.g., any suitable reverse transcriptase known in the art). In some embodiments, adding or incorporating the polynucleotide sequence to the 3′ end of the extension product in step (c) includes the use of the reverse transcriptase. In some embodiments, adding or incorporating the polynucleotide sequence to the 3′ end of the extension product in step (c) includes the use of a terminal transferase. In some embodiments, the terminal transferase is terminal deoxynucleotidyl transferase. In some embodiments, the reverse transcriptase or the terminal transferase adds or incorporates at least three nucleotides to the extension product. In some embodiments, the reverse transcriptase or the terminal transferase adds or incorporates 4, 5, 6, 7, 8, 9, 10, or more nucleotides (e.g., a polynucleotide sequence) to the 3′ end of the extension product(s) in step (c). In some embodiments, the polynucleotide sequence added or incorporated to the 3′ end of the extension product(s) is 5′-CCC-3′. In some embodiments, the polynucleotide sequence added or incorporated to the 3′ end of the extension product(s) is 5′-CGC-3′.

In some embodiments, the extending in step (d) includes using the second primer as a template. In some embodiments, the second primer includes RNA.

In some embodiments, a second primer is added before, contemporaneously with, or after reverse transcription or other terminal transferase-based reactions. In certain embodiments, methods of biological sample analysis using a second primer can involve the generation of nucleic acid products from target nucleic acids of the biological sample, followed by further processing of the nucleic acid products with the second primer.

In some embodiments, the method includes removing the target nucleic acid, or any other nucleic acid hybridized to the extension product(s) (e.g., extended cDNA product) before the complement of the capture sequence of the extension product(s) hybridizes to the capture domain of the capture probe. In some embodiments, the method includes removing the target nucleic acid and the ribo-second primer hybridized to the extension product (e.g., extended cDNA product) before the capture sequence of the second primer hybridizes to the capture domain of the capture probe. In some embodiments, the removing includes the use of an RNase. In some embodiments, the RNase is RNase A. In some embodiments, the RNase is RNase P. In some embodiments, the RNase is RNase Ti. In some embodiments, the RNase is RNase H. In some embodiments, the removing includes use of heat.

In some embodiments, the method includes removing the target nucleic acid and/or any other nucleic acid hybridized to the extension product (e.g., extended DNA product) before the complement of the capture sequence of the second primer (incorporated into the extension product) hybridizes to the capture domain of the capture probe.

In some embodiments, the extension product(s) (e.g., the single-stranded extension product(s)) hybridizes to the capture domain of the capture probe on the substrate. In some embodiments, the 3′ end of the capture probe is extended using the extension product as a template. In some examples, the 3′ end of the extension product (e.g., single-stranded cDNA product) is extended using the capture probe as a template, thereby generating an extended capture product. In some embodiments, both the capture probe, and the extension products hybridized thereto, are extended from their 3′ ends. In some embodiments, the extending includes the use of a polymerase. Any suitable polymerase can be used (e.g., Kapa HiFi). In some examples, the 3′ end of the capture probe is extended using the extension product as a template and the 3′ end of the extension product is simultaneously extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

In some embodiments, the released, extended captured products can be prepared for downstream applications, such as generation of a sequencing library and next-generation sequencing. Generating sequencing libraries are known in the art. For example, the extended captured products can be purified and collected for downstream amplification steps. The extended amplification products can be amplified using PCR, where primer binding sites flank the spatial barcode and target nucleic acid, or a complement thereof, generating a library associated with a particular spatial barcode. In some embodiments, the library preparation can be quantitated and/or quality controlled to verify the success of the library preparation steps. The library amplicons are sequenced and analyzed to decode spatial information and the target nucleic acid sequence.

Alternatively or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flowcell (e.g., on Illumina sequencing instruments). Additionally, i7 and i5 can index sequences be added as sample indexes if multiple libraries are to be pooled and sequenced together. Further, Read 1 and Read 2 sequences can be added to the library preparation for sequencing purposes. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

In some embodiments, the biological sample is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the first and/or second primer to the biological sample.

In some embodiments, the method includes staining the biological sample. In some embodiments, the staining includes the use of hematoxylin and/or eosin. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The biological sample can be stained using known staining techniques, including Can-Grunwald, Giemsa, hematoxylin and/or eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, the method includes a step of permeabilizing the biological sample (e.g., a tissue section). For example, the biological sample can be permeabilized to facilitate transfer of the extended products to the capture probes on the array. In some embodiments, the permeabilizing includes the use of an organic solvent (e.g., acetone, ethanol, and methanol), a detergent (e.g., saponin, Triton X-100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™ Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, the entire contents of which are incorporated herein by reference.

In some embodiments, the biological sample is a tissue sample. In some embodiments, the biological sample (e.g., tissue sample) is a tissue microarray. A tissue microarray contains multiple representative tissue samples from different cases assembled on a single histologic slide, and therefore allows high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

In some embodiments, the tissue sample is a fixed tissue sample. For example, fixing the biological sample can include the use of a fixative including: ethanol, methanol, acetone, formaldehyde, paraformaldehyde-Triton, glutaraldehyde, and combinations thereof. In some embodiments, the biological sample can be fixed using PAXgene. For example, the biological sample can be fixed using PAXgene in addition, or alternatively to, a fixative disclosed herein or known in the art (e.g., an alcohol such as ethanol or methanol, acetone, acetone-alcohol, formalin, paraformaldehyde, paraformaldehyde-Triton, glutaraldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which are hereby incorporated by reference in their entirety, for a description and evaluation of PAXgene for tissue fixation.

In some embodiments, the fixed tissue sample is a formalin-fixed paraffin embedded tissue sample, paraformaldehyde-fixed tissue sample, a methanol-fixed tissue sample, or an acetone-fixed tissue sample. In some embodiments, the tissue sample is a fresh frozen tissue sample. In some embodiments, the biological sample is a tissue section. In some embodiments, the biological sample is a fixed tissue section (e.g., a fixed tissue section prepared by any of the methods described herein).

The tissue sample can be obtained from any suitable location in a tissue or organ of a subject, e.g., a human subject. In some embodiments, the tissue sample is a mouse sample. In some embodiments, the tissue sample is a human sample. In some embodiments, the tissue sample can be derived from skin, brain, breast, lung, liver, kidney, prostate, tonsil, thymus, testes, bone, lymph node, ovary, eye, heart, or spleen (e.g., from mouse or human). In some embodiments, the tissue sample is derived from normal or diseased tissue. In some embodiments, the sample is an embryo sample. The embryo sample can be a non-human embryo sample. In some instances, the sample is a mouse embryo sample.

In some embodiments, the method includes generating a sequencing library. In some embodiments, the determining in step (f) includes sequencing. Methods and systems for sequencing are known in the art and are described herein. In some embodiments, the sequencing is high-throughput sequencing.

### Enrichment or Depletion

In some embodiments, the methods described herein further include enrichment and/or depletion steps. This can be advantageous to improve the spatial analysis of target nucleic acids (e.g., mRNAs encoding immune cell receptors) from a biological sample.

For example, targeted RNA depletion can allow for depletion or removal of one or more species of undesirable RNA molecules (e.g., ribosomal RNA and/or mitochondrial RNA), thereby reducing the pool and concentration of undesirable RNA molecules in the sample which could interfere with desired target detection (e.g., detection of mRNA encoding an immune cell receptor). Examples of the undesirable RNA include, but are not limited to, messenger RNA (mRNA), ribosomal RNA (rRNA), mitochondrial RNA (mtRNA), transfer RNA (tRNA), microRNA (miRNA), and viral RNA. In some embodiments, to achieve depletion, one or more probes can be used that selectively hybridize to undesirable RNA (e.g., ribosomal RNA or mitochondria RNA), thereby reducing the pool and concentration of undesirable RNA in the sample. Subsequent application of capture probes to the sample can result in improved capture of other types of target RNA due to a reduction in undesirable RNA (e.g., down-selected RNA) present in the sample. This can be advantageous, for example, when random hexamers or decamers are used during the reverse transcription reaction, since removal of ribosomal RNA using a negative selection using undesirable RNA probes (e.g., biotinylated baits against ribosomal RNA) can improve the signal to noise.

Alternatively or additionally, in some embodiments, primers against sequences encoding the constant region of an immune cell receptor can be used for downstream enrichment.

### Sandwich Configurations

Some steps of the methods described herein can be performed in a biological sample (e.g., in situ) prior to contacting the biological sample with the array including a plurality of capture probes. In some embodiments, the biological sample is disposed or placed on the array including the plurality of capture probes prior to step (a). In some embodiments, the biological sample is disposed or placed on the array including the plurality of capture probes prior to step (f). In some embodiments, the biological sample is not disposed or placed on the array. For example, the biological sample can be placed on a substrate (e.g., a slide) that does not include a spatial array. In some embodiments, the substrate including the biological sample can be aligned with the array (e.g., “sandwiched”) such that at least a portion of the biological sample is aligned with at least a portion of the array. In embodiments where the biological sample is disposed or placed on a substrate, steps (a)-(e) can be performed prior to aligning the substrate with the array as described herein. In embodiments where the biological sample is disposed or placed on a substrate, steps (a)-(e) can be performed after aligning the substrate with the array as described herein.

In some embodiments one or more extension products generated from the biological sample are released from the biological sample and migrate to a substrate comprising an array of capture probes where the extension product(s) hybridize to the capture probes of the array. In some embodiments, the release and migration of the extension product(s) to the substrate comprising the array of capture probes occurs in a manner that preserves the original spatial context of the extension product(s) in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., U.S. Patent Application Pub. No. 20210189475, WO 2021/252747, and WO 2022/061152A. In some embodiments, the sandwiching process may be facilitated by a device, sample holder, sample handling apparatus, or system described in, e.g., U.S. Patent Application Pub. No. 20210189475, WO 2021/252747, or WO 2022/061152A.

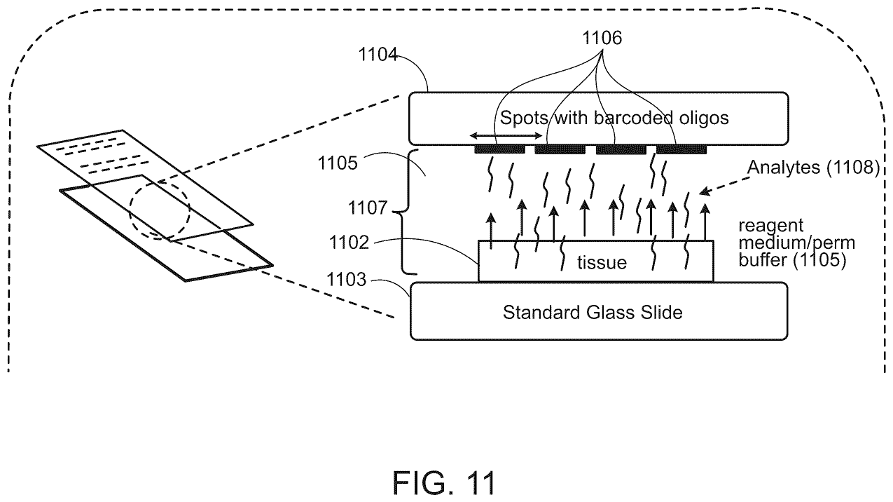

FIG. 11 is a schematic diagram depicting an exemplary sandwiching process 1104 between a first substrate comprising a biological sample (e.g., a tissue section 1102 on a slide 1103) and a second substrate comprising a spatially barcoded array, e.g., a slide 1104 that is populated with spatially-barcoded capture probes 1106. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 1104) is in a superior position to the first substrate (e.g., slide 1103). In some embodiments, the first substrate (e.g., slide 1103) may be positioned superior to the second substrate (e.g., slide 1104). A reagent medium 1105 (e.g., permeabilization solution) within a gap 1107 between the first substrate (e.g., slide 1103) and the second substrate (e.g., slide 1104) creates a permeabilization buffer which permeabilizes or digests the biological sample 1102 and the extension product(s) 1108 generated in the biological sample 1102 may release, actively or passively migrate (e.g., diffuse) across the gap 1107 toward the capture probes 1106, and hybridize to the capture probes 1106.

After the extension product(s) 1108 hybridize to the capture probes 1106, an extension reaction may occur, thereby generating a spatially barcoded library. A polymerase can be used to generate a cDNA library associated with a particular spatial barcode. Barcoded cDNA libraries can be mapped back to a specific spot on a capture area of the capture probes 1106. This data can be subsequently layered over a high-resolution microscope image of the biological sample, making it possible to visualize the data within the morphology of the tissue in a spatially-resolved manner. In some embodiments, the extension reaction can be performed separately from the sample handling apparatus described herein that is configured to perform the exemplary sandwiching process 1104. The sandwich configuration of the sample 1102, the first substrate (e.g., slide 1103) and the second substrate (e.g., slide 1104) can provide advantages over other methods of spatial analysis and/or analyte capture or proxies thereof (e.g., extension product(s)). For example, the sandwich configuration can reduce a burden of users to develop in house tissue sectioning and/or tissue mounting expertise. Further, the sandwich configuration can decouple sample preparation/tissue imaging from the barcoded array (e.g., spatially-barcoded capture probes 1106) and enable selection of a particular region of interest of analysis (e.g., for a tissue section larger than the barcoded array). The sandwich configuration also beneficially enables spatial analysis without having to place a biological sample (e.g., tissue section) 1102 directly on the second substrate (e.g., slide 1104).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the extension product(s) from the biological sample.

The sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to a to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

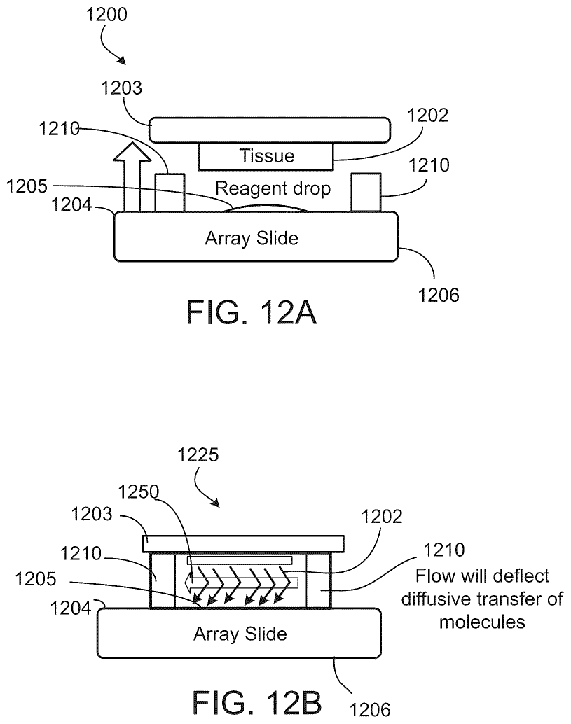

FIG. 12A shows an exemplary sandwiching process 1200 where a first substrate (e.g., slide 1203), including a biological sample 1202 (e.g., a tissue section), and a second substrate (e.g., slide 1204 including spatially barcoded capture probes 1206) are brought into proximity with one another. As shown in FIG. 12A a liquid reagent drop (e.g., permeabilization solution 1205) is introduced on the second substrate in proximity to the capture probes 1206 and in between the biological sample 1202 and the second substrate (e.g., slide 1204 including spatially barcoded capture probes 1206). The permeabilization solution 1205 can release extension product(s) that can be captured (e.g., hybridized) by the capture probes 1206 of the array. As further shown, one or more spacers 1210 can be positioned between the first substrate (e.g., slide 1203) and the second substrate (e.g., slide 1204 including spatially barcoded capture probes 1206). The one or more spacers 1210 may be configured to maintain a separation distance between the first substrate and the second substrate. While the one or more spacers 1210 is shown as disposed on the second substrate, the spacer may additionally or alternatively be disposed on the first substrate.

FIG. 12B shows a fully formed sandwich configuration creating a chamber 1250 formed from the one or more spacers 1210, the first substrate (e.g., the slide 1203), and the second substrate (e.g., the slide 1204 including spatially barcoded capture probes 1206) in accordance with some example implementations. In FIG. 12B, the liquid reagent (e.g., the permeabilization solution 1205) fills the volume of the chamber 1250 and can create a permeabilization buffer that allows extension product(s) to diffuse from the biological sample 1202 toward the capture probes 1206 of the second substrate (e.g., slide 1204). In some aspects, flow of the permeabilization buffer may deflect extension product(s) from the biological sample 1202 and can affect diffusive transfer of extension product(s) for spatial analysis. A partially or fully sealed chamber 1250 resulting from the one or more spacers 1210, the first substrate, and the second substrate can reduce or prevent flow from undesirable convective movement of transcripts and/or molecules over the diffusive transfer from the biological sample 1202 to the capture probes 1206.

### Compositions

Also provided herein are compositions including a) a target nucleic acid; and b) one or more extension products hybridized to the target nucleic acid, where the one or more extension products includes in a 5′ to 3′ direction: i) a primer hybridized to the target nucleic acid in a region encoding a constant region of an immune cell receptor (e.g., B cell receptor or a T cell receptor), where the primer comprises a functional domain; ii) a sequence substantially complementary to a region of the target nucleic acid that encodes the variable region of an immune cell receptor (e.g., V and/or J sequences) of the immune cell receptor (e.g., B cell receptor or T cell receptor); and iii) a polynucleotide sequence (e.g., a non-templated sequence that is at least three nucleotides in length).

In some embodiments, the composition includes a second primer hybridized to the polynucleotide sequence, where the second primer includes a capture sequence. In some embodiments, the extension product includes a complement of the capture sequence.

In some embodiments, the target nucleic acid is RNA. In some embodiments, the RNA is mRNA. In some embodiments, the mRNA includes a sequence encoding an antibody or a fragment thereof. In some embodiments, the mRNA includes a sequence encoding a T cell receptor or a fragment thereof. In some embodiments, the mRNA comprises a sequence encoding a B cell receptor.

In some embodiments, the functional domain includes a primer binding site. In some embodiments, the functional domain includes a sequencing specific site.

In some embodiments, the second primer includes RNA.

In some embodiments, the composition includes a reverse transcriptase. In some embodiments, the composition includes a helicase. In some embodiments, the composition includes one or more single-stranded DNA binding protein selected from the group consisting of: Tth RecA, E. coli RecA, T4 gp32 and ET-SSB. In some embodiments, the composition includes a superhelicase selected from the group consisting of: Rep, PrcA, UvrB, RecBCD, and Tte-Uvrd.

In some embodiments, the polynucleotide sequence includes a homopolynucleotide sequence. In some embodiments, the polynucleotide sequence includes a heteropolynucleotide sequence.

In some embodiments, the extension products further include a sequence substantially complementary to a region of the target nucleic acid that encodes a D sequence of an immune cell receptor.

Also provided herein are compositions including: one or more extension products, where the one or more extension products include in a 5′ to 3′ direction: i) a primer, where the primer includes a functional domain; ii) a sequence substantially complementary to a region of a target nucleic acid that encodes V and J sequences of an immune cell receptor (e.g., a B cell receptor or a T cell receptor); and iii) a polynucleotide sequence, where the one or more extension products is hybridized to a capture domain on an array, where the array includes a plurality of capture probes, and where a capture probe of the plurality of capture probes includes a spatial barcode and the capture domain.

In some embodiments, the polynucleotide sequence is a heteropolynucleotide sequence. In some embodiments, the polynucleotide sequence is a homopolynucleotide sequence.

In some embodiments, the capture probe includes a cleavage domain, one or more functional domains, a unique molecular identifier, or a combination thereof.

In some embodiments, the functional domain includes a primer binding sequence or a sequencing specific sequence.

In some embodiments, the one or more extension products include a sequence substantially complementary to a region of the target nucleic acid that encodes a D sequence of an immune cell receptor.

In some embodiments, the target nucleic acid is RNA. In some embodiments, the RNA is mRNA.

In some embodiments, the primer includes a homopolymer sequence and a functional domain. In some embodiments, the homopolymer sequence is a poly(T) sequence. In some embodiments, the first primer includes a random sequence and a functional domain. In some embodiments, the random sequence includes 5, 6, 7, 8, 9, 10, 11, 12, 13, 14, 15, 16, 17, 18, 19, 20, or more random nucleotides. In some embodiments, the random sequence is a random hexamer. In some embodiments, the random sequence is a random decamer. In some embodiments, the functional domain includes a primer binding site. In some embodiments, the functional domain includes a sequencing specific site.

### Kits

Also provided herein are kits including: (a) an array including a plurality of capture probes, where a capture probe of the plurality of capture probes includes: (i) a spatial barcode; and (ii) a capture domain that binds a capture sequence, or a complement thereof; (b) a first primer including a sequence substantially complementary to a target nucleic acid and a functional domain, where the functional domain includes a primer binding sequence or a sequencing specific sequence; (c) a second primer including a capture sequence; and optionally (d) instructions for performing any of the methods described herein.

In some embodiments, the capture probe includes one or more functional domains, a unique molecular identifier, a cleavage domain, or a combination thereof.

In some embodiments, the kit includes a reverse transcriptase. In some embodiments, the reverse transcriptase has strand displacement activity. In some embodiments, the kit includes a terminal deoxynucleotidyl transferase. In some embodiments, the kit includes a polymerase.

In some embodiments, the capture domain includes a fixed sequence as described herein.

In some embodiments, the second primer is RNA.

In some embodiments, the kit includes a helicase. In some embodiments, the kit includes one or more single-stranded DNA binding proteins such as one or more of: Tth RecA, E. coli RecA, T4 gp32 and ET-SSB. In some embodiments, the kit includes a superhelicase. The superhelicase can be selected from the group consisting of: Rep, PrcA, UvrB, RecBCD, and Tte-Uvrd.

In some embodiments, the kit includes one or more hybridization probes, wherein a hybridization probe includes (i) a sequence substantially complementary to a nucleic acid encoding an immune cell receptor and (ii) a binding moiety (e.g., biotin) that interacts with a capturing moiety (e.g., avidin, streptavidin). Also provided herein are kits, including one or both of ribosomal RNA depletion probes and mitochondrial RNA depletion probes.

In some embodiments, the kit includes instructions for performing any of the methods described herein.

### Exemplary Applications

Also provided herein are uses for the disclosed methods. For example, provided herein are methods for determining the presence and/or abundance of an immune cell clonotype at a location in a biological sample. Some embodiments of any of the methods described herein include capturing transcripts to identify an immune cell clonotype. Thus, the disclosed methods of 5′ capture of target nucleic acids can be used for determining the presence and/or abundance of an immune cell clonotype at a location in a biological sample. Identifying the clonal regions, e.g., regions defined by the places where variable (V), diverse (D), and/or joining (J) segments join to from the complementarity determining regions, including CDR1, CDR2, and CDR3, which provide specificity to the TCRs and/or BCRs, and by coupling clonal information to spatial information, it is possible to understand which T-cell and B-cell clonotypes may be specifically interacting with given cell types within a biological sample.

The disclosed methods are also useful in a clinical setting (e.g., monitoring minimal residual disease (MRD)). Patients treated for many cancers often retain an MRD related to the cancer. That is, even though a patient may have by clinical measures a complete remission of the disease in response to treatment, a small fraction of the cancer cells may remain that have escaped destruction. The type and size of this residual population can be an important prognostic factor for the patient's continued treatment. Thus, the more sensitive the measurement of MRD, the more likely that a subsequent course of treatment will be successful.

In particular, a feature of certain acute lymphoblastic leukemias (ALLs) is the sequence evolution of clonotypes associated with the disease. Although treatment outcomes in ALL have improved dramatically over the past fifty years, a significant proportion of patients will ultimately relapse, usually with disease that is highly refractory to additional therapy. It is generally thought that these relapses are due to residual leukemic cells that are resistant to therapy and remain undetected during clinical remission. Clonality of B-cell populations can be assessed by analysis of gene rearrangements that occur at the immunoglobulin heavy chain (IgH) gene locus and the resulting unique VDJ rearrangements are used as clonotypic markers in precursor-B-cell ALL. Precursor-B-cell ALL is generally thought to be a clonal disease resulting from malignant transformation and expansion of a single B-cell; however, changes in clonal IgH rearrangements between initial diagnosis and relapse has been shown in a significant proportion of pre-B ALL cases. Thus, the disclosed methods can be used for diagnosing and monitoring sequence evolution of IgH clonotypes in ALL, particularly precursor B cell ALL.

In many malignant lymphoid and myeloid neoplasms, a diagnostic tissue sample, such as a peripheral blood sample or a bone marrow sample, is obtained before treatment from which a clonotype profile is generated (a diagnostic clonotype profile). One or more disease-correlated clonotypes are identified in the clonotype profile, usually as the clonotypes having the highest frequencies, e.g., greater than 5 percent. After treatment, the presense, absence, or frequency of such correlating clonotypes is assessed periodically to determine whether a remission is holding or whether the neoplasm is returning or relapsing, based on the presence of, or an increase in the frequency of, the correlating clonotypes (or related clonotypes) in a post-treatment clonotype profile. That is, after treatment, minimal residual disease of the cancer is assessed based on the presence, absence, or frequency of the correlating clonotypes and/or related clonotypes, such as clonotypes evolved therefrom by further substitution, or other mechanisms. In one aspect of the invention, a measure of MRD is taken as a frequency of the one or more clonotypes initially identified as being correlated with the cancer together with the clonotypes evolved therefrom after such initial identification.

Provided herein is a method of monitoring minimal residual disease of a B cell leukemia (e.g., acute lymphoblastic leukemia) patient by assaying one or more patient-specific immune cell clonotypes correlated with the B cell leukemia. In some embodiments, the method includes (a) obtaining from the patient a biological sample comprising B-cells; and (b) determining from the biological sample a presence, absence and/or level of one or more patient-specific immune cell clonotypes correlated with the B cell acute lymphoblastic leukemia and/or previously unrecorded clonotypes evolved therefrom. The method can include repeating step (b) one or more times on a successive sample from the patient to monitor the B cell acute lymphoblastic leukemia. In some embodiments, determining the presence, absence and/or level of one or more patient-specific immune cell clonotypes is performed in accordance with any method of determining a location of a target nucleic acid disclosed herein, including methods of 5′ capture of target nucleic acids.

In some aspects, the method includes monitoring and treating a patient. For example, in some embodiments, the methods of monitoring minimal residual disease further include a step of modifying a treatment regimen of the patient based on a presence, absence and/or level of the one or more patient-specific clonotypes and clonotypes related thereto. The patient can be treated in accordance with suitable treatment regimens in the art. After treatment, the presence, absence or frequency of correlating clonotypes is assessed periodically to determine whether a remission is holding or whether the neoplasm is returning or relapsing, based on the presence of, or an increase in the frequency of, the correlating clonotypes (or related clonotypes) in a post-treatment clonotype profile. That is, after treatment, minimal residual disease of the cancer is assessed based on the presence, absence or frequency of the correlating clonotypes and/or related clonotypes. In one aspect of the invention, a measure of MRD is taken as a frequency of the one or more clonotypes initially identified as being correlated with the cancer together with the clonotypes evolved therefrom after such initial identification. If increased numbers of leukemia cells are detected (e.g. between successive MRD measurements), then a relapse has taken place and the treatment regimen is modified to regain a remissive state. The modification may include use of a different chemotherapeutic combination, use of a different administration schedule, use of different amounts of drug, or a switch to a differ kind of therapy.

### Example 1. 5′ Capture of Target Nucleic Acids

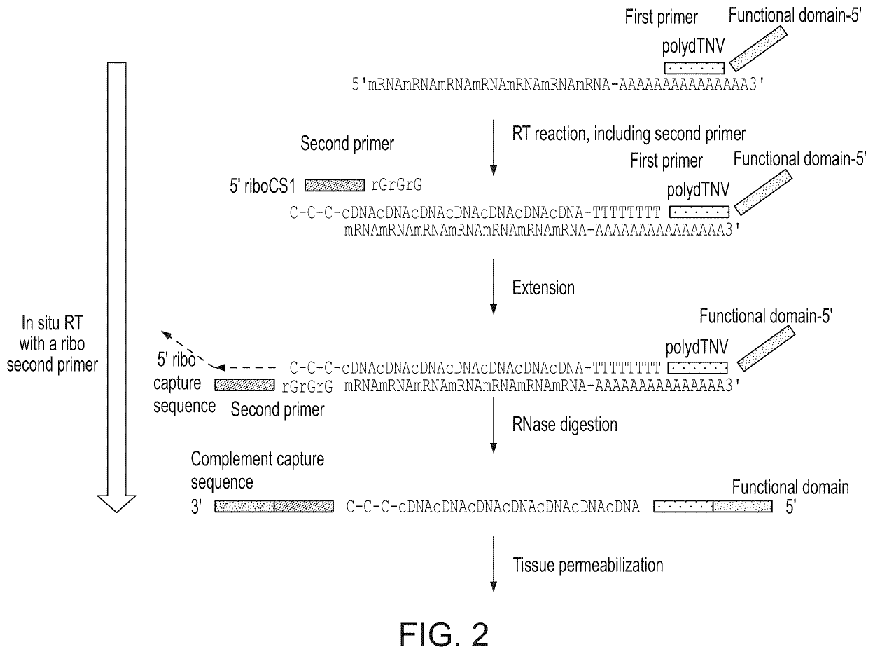

FIG. 2 is a schematic showing generation of a cDNA by in situ reverse transcription of a target nucleic acid (e.g., mRNA) from a first primer including a sequence substantially complementary to the target nucleic acid and a functional domain and a second primer that includes a capture sequence and a sequence substantially complementary to a homopolynucleotide sequence.

More specifically, target nucleic acids are contacted with a first primer that includes a sequence substantially complementary to the target nucleic acid (e.g., poly(dT) sequence, a poly(dTNV) sequence, a random sequence, a sequence encoding a constant region of a B cell receptor (e.g., an antibody), or a T cell receptor) and a functional domain. In some examples, the functional domain is a primer binding site. In some examples, the functional domain is a sequencing specific site (e.g., Read 2 site). The target nucleic acid is reverse transcribed into cDNA and a polynucleotide sequence is added to the 3′ end of the cDNA. FIG. 2 shows a homopolynucleotide sequence comprising cytosines, however, it is appreciated that other polynucleotide sequences can be added to the 3′ end of the cDNA, including a heteropolynucleotide sequence. In some examples, the polynucleotide sequence is added by the reverse transcriptase. In some examples, the polynucleotide sequence is added by a terminal transferase (e.g., terminal deoxynucleotidyl transferase).

A second primer is added where the second primer includes a sequence substantially complementary to the polynucleotide (e.g., a homopolynucleotide sequence, a heteropolynucleotide sequence) sequence added to the 3′ end of the cDNA and a capture sequence. In some examples, the second primer is RNA. After reverse transcription and extension of the 3′ end of the cDNA using the second primer as an extension template, an RNase (e.g., RNase H) is contacted with the biological sample (e.g., a tissue section). The RNase degrades the RNA strand of the RNA/cDNA duplex, leaving a single-stranded cDNA product (e.g., an extension product) that includes the first primer at its 5′ end and the complement of the capture sequence capable of hybridizing a capture domain of a capture probe.

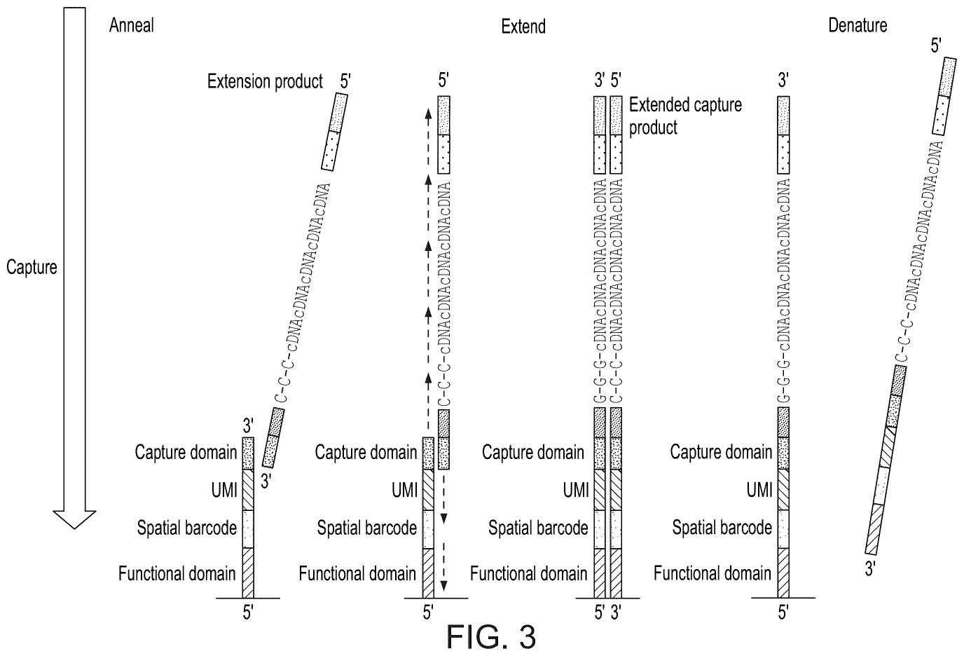

FIG. 3 is a schematic showing capture of the extension product (e.g., the single-stranded cDNA product shown in FIG. 2) by a capture probe on the substrate. The capture probe is attached to the substrate via its 5′ end and can include one or more functional domains, a spatial barcode, a unique molecular identifier, a capture domain, or a combination thereof. In some examples, the capture probe also includes a cleavage domain. The capture domain hybridizes to the complement of the capture sequence within the extension product (e.g., single-stranded cDNA product) from FIG. 2. In some examples, the 3′ end of the capture probe is extended using the extension product as a template. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

FIG. 4 is a schematic diagram showing an embodiment of FIG. 2 where reverse transcription of target nucleic acids is performed with a plurality of primers. In some examples, reverse transcription is performed using a reverse transcriptase with strand displacement activity. In some examples, reverse transcription is performed with a reverse transcriptase and a helicase. In some examples, reverse transcription is performed with a reverse transcriptase and a superhelicase. In some examples, reverse transcription is performed with one or more single-stranded DNA binding proteins. When a plurality of primers are used to template reverse transcription as shown in FIG. 4, the resulting extension products can be of different lengths depending on where the primer hybridized to the target nucleic acid. In some examples, a primer of the plurality of primers can facilitate more than one reverse transcription reaction, thus resulting in two or more extension products generated from the same primer.

The released, extended captured products can be prepared for downstream applications, such as generation of a sequencing library and next-generation sequencing as described herein.

### Example 2. 5′ Capture of Target Nucleic Acids

A fresh frozen mouse brain sample was sectioned and placed on an array slide containing capture probes having a blocked capture domain. The tissue sections were fixed 5 minutes in 4% formaldehyde, followed by 5 minutes of decrosslinking in 0.iN HCl.

The sections were washed in 1× PBS and reverse transcription (RT) was performed using a polydT30NV primer and an in-house reverse transcriptase (RT) enzyme at 42° C. overnight. Fluorescently labeled Cy3-dCTPs were spiked into the RT buffer to permit visualization of the synthesized cDNA. Additionally, a template switching ribonucleotide (rTSO) having a capture sequence as a handle was spiked in to allow the incorporation of the handle into the cDNA.

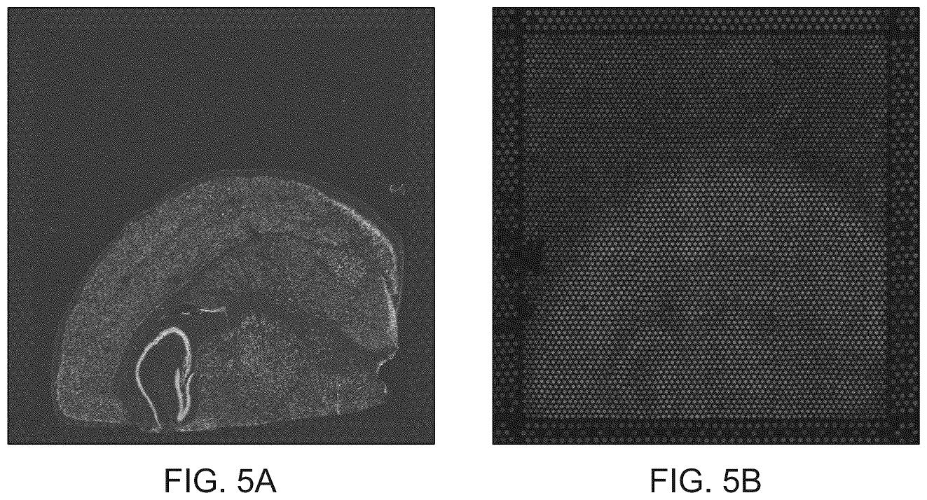

The next day, the sections were washed in 0.2×SSC/20% Ethylene Carbonate at 50° C. to remove any non-specific signal for the array. Afterwards the sections were imaged under the microscope (Cy3 channel, FIG. 5A). Post imaging, the RNA was digested using RNaseH, followed by tissue permeabilization.

Post permeabilization, the bound cDNA was extended using a polymerase and Cy3 was spiked into the mixture. Post extension, the slides were washed in 2×SSC followed by imaging (FIG. 5B).

FIGS. 5A-B are mouse brain images showing fluorescently labeled cDNA post reverse transcription (FIG. 5A) in situ where the reverse transcription reaction was performed overnight at 42° C. with Cy3 labeled dCTP and results in a tissue “footprint” (e.g., the fluorescently labeled cDNA reproduces the morphological characteristics of the tissue section). FIG. 5B shows fluorescently labeled extended cDNA post permeabilization and cDNA extension which also results in a tissue footprint.

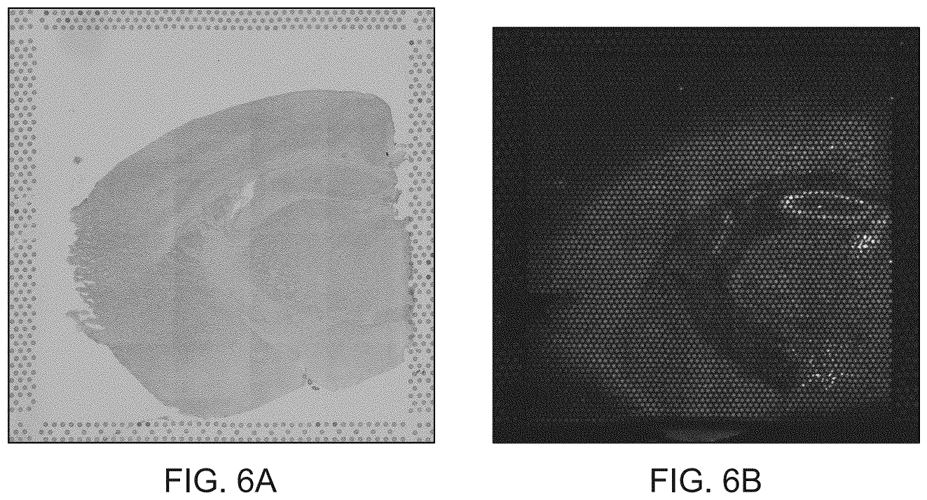

FIGS. 6A-B show mouse brain images from experiments similar to that described in FIGS. 5A-5B, but the RT reaction was performed without Cy3-dCTP spike in. FIG. 6A shows a brightfield image of a mouse brain tissue section and FIG. 6B shows fluorescently labeled extended cDNA where the capture domain of the capture probe is blocked. During extension, Cy3-dCTPs were spiked in to permit visualization of the captured cDNA.

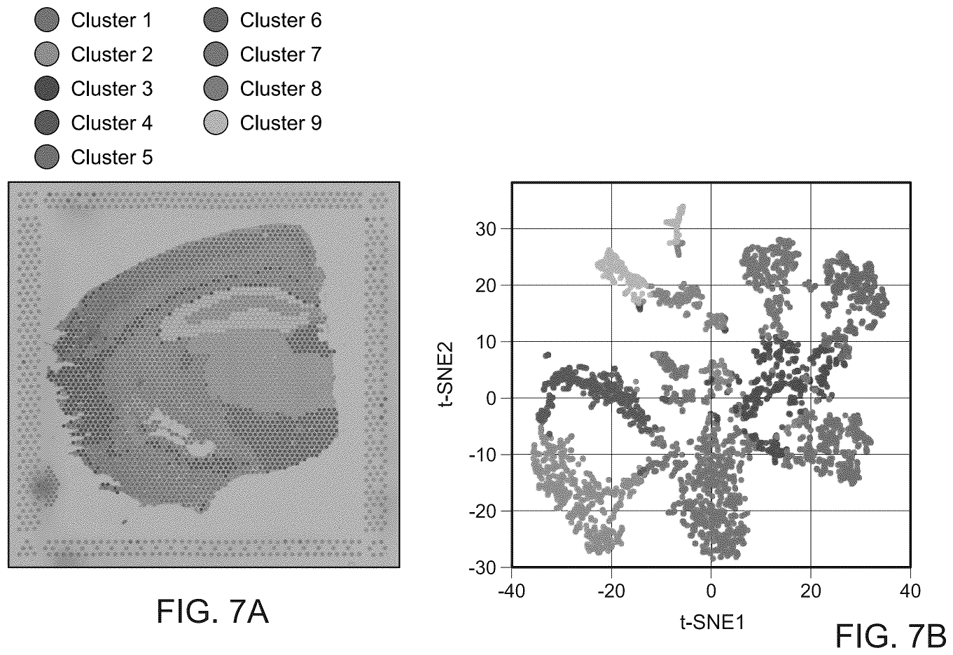

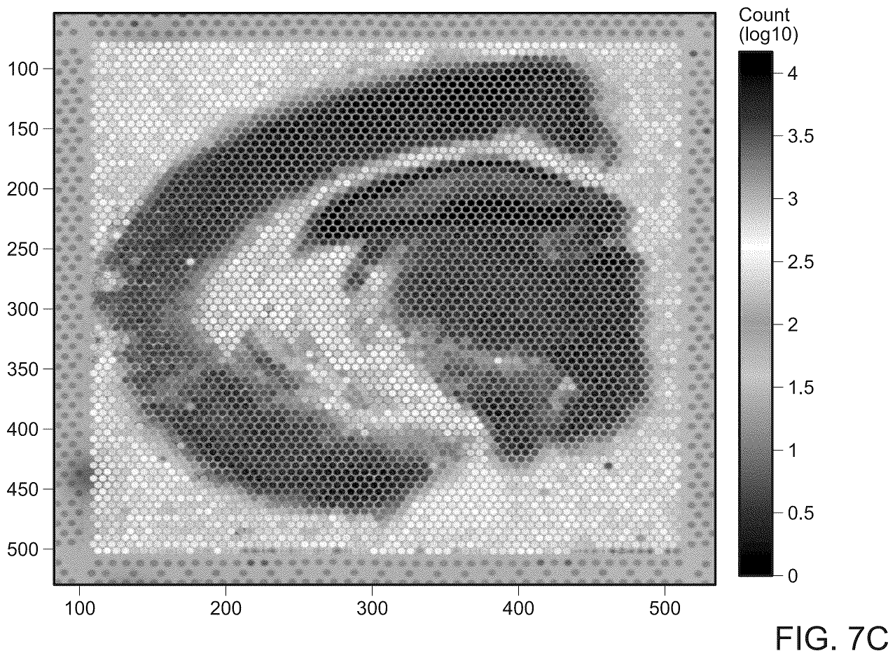

FIG. 7A shows spatial gene expression clusters, the corresponding t-SNE plot (FIG. 7B), and spatial gene expression heat map (FIG. 7C) from capture of extension products generated from experiments as described for FIGS. 5A-5B, except that RT and extension were performed without any Cy3-dCTP spike-in. The captured and extended cDNA was released using 0.08N KOH, followed by standard library preparation for next generation sequencing.

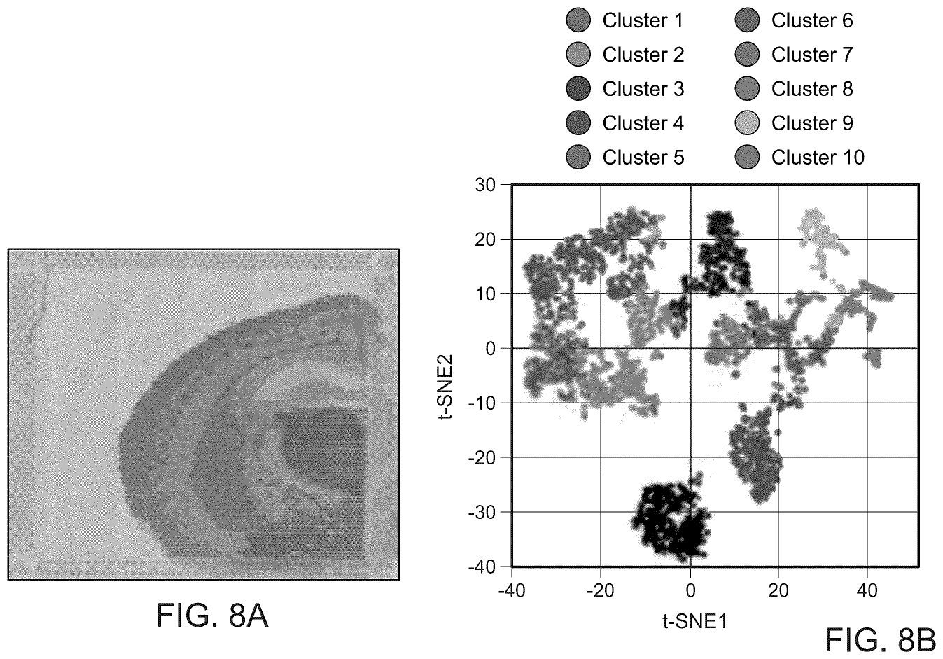

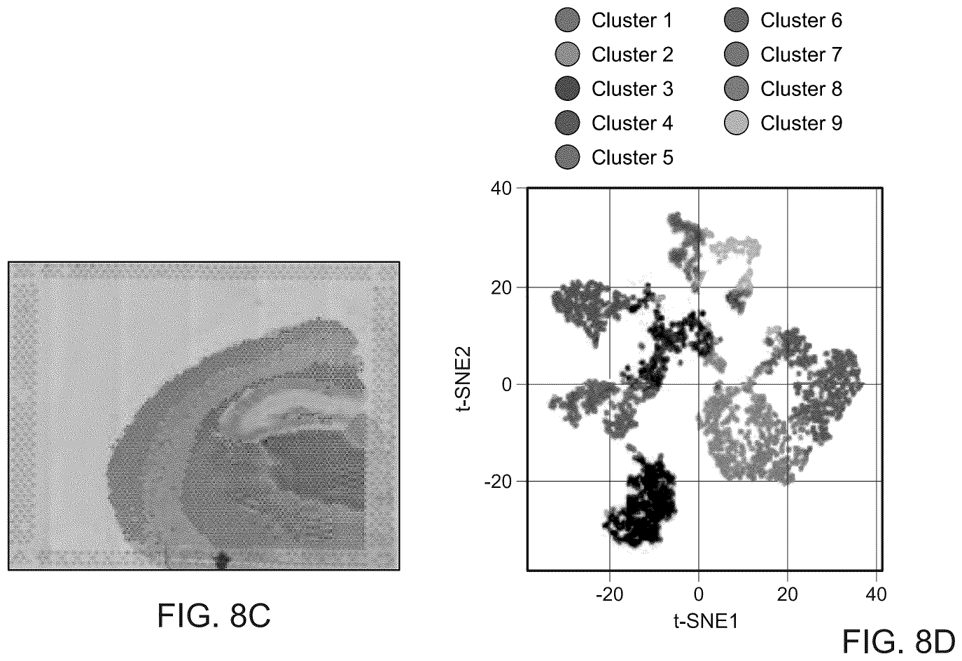

FIGS. 8A-D show spatial gene expression clustering with a first primer including a poly(T) sequence (e.g., poly(T)30NV) (FIG. 8A) and the corresponding t-SNE plot (FIG. 8B) and spatial gene expression clustering with a first primer including a random decamer (FIG. 8C) and the corresponding t-SNE plot (FIG. 8D) demonstrating that spatial gene expression information can be captured with in situ amplification with a first primer including either a poly(T) sequence or a random decamer sequence and where a complement of a capture sequence is incorporated into extension product(s) described herein.

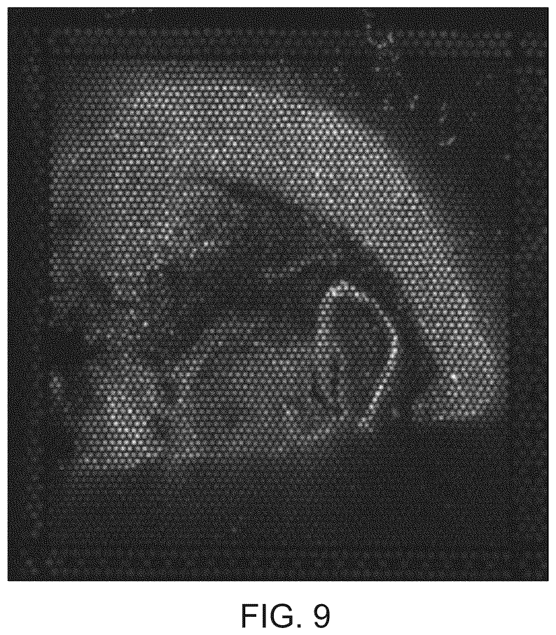

FIG. 9 shows fluorescently labeled extended probes captured in mouse brain tissue using an alternative capture sequence as the handle of the TSO, thereby demonstrating that in situ template switching functions with various sequence handles.

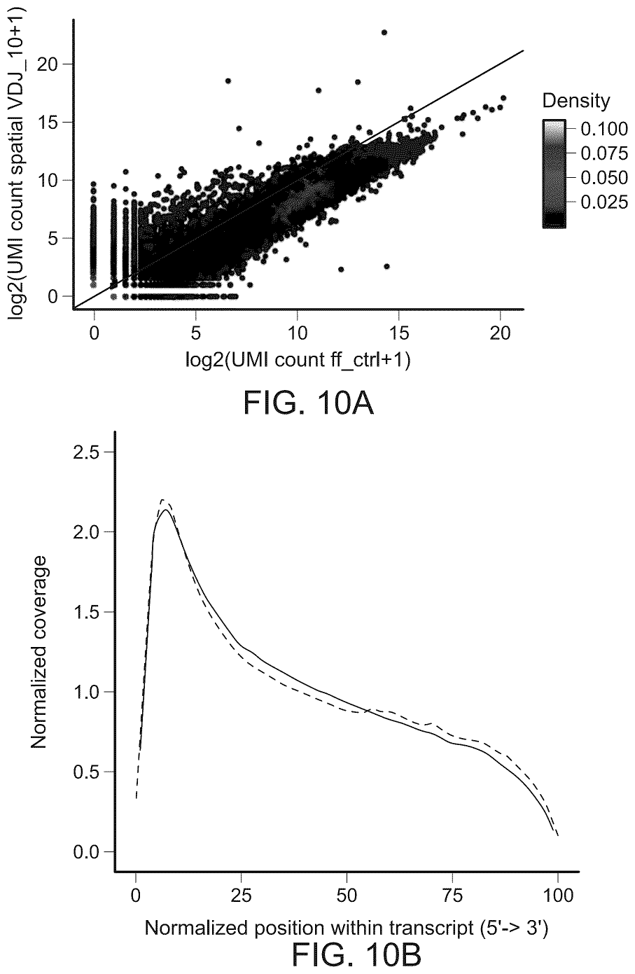

FIGS. 10A-B are graphs showing correlation between fresh frozen capture using standard Visium spatial gene expression (10×Genomics) and spatial 5′ end capture using the methods disclosed herein (FIG. 10A). Each dot represents the UMI counts for a single gene. FIG. 10B is a graph showing the normalized position of each mapped read within the full-length transcript. The data shown in the graph confirms successful 5′ capture of transcripts.

The methods described herein are also able to identify sequences encoding for a complementarity determining region (“CDR”) e.g., CDR1, CDR2, and/or CDR3 sequences. Determining the sequence of CDRs can also identify clonotypes within a biological sample. Table 1 below shows CDR3 sequences identified from a biological sample. The sequences were obtained from an experiment using fresh frozen Jurkat cell pellets. Jurkat cells express a defined T cell receptor alpha chain (TRA) and T cell receptor beta chain (TRB). The 5′ capture assay was performed with downstream enrichment of VDJ sequences. For this enrichment, primers from single cell 5′GEX Immunoprofiling solution from 10× Genomics were used, followed by library preparation. To analyze the TRA and TRB sequences, the single cell VDJ pipeline (Cell Ranger VDJ) from 10× Genomics was used. The identified TRA and TRB sequences match the expected sequence from the Jurkat reference.

TABLE 1

| Clonotype | SEQ ID | | | | |
|---|---|---|---|---|---|
| ID | NO. | CDR3 | Sequence | Frequency | Proportion |
| 1 | SEQ ID | TRB | CASSFSTCSANYGYTF | 478 | 95.98% |
| | NO: 1 | TRA | CAVSDLEPNSSASKIIF | | |
| | SEQ ID | | | | |
| | NO: 2 | | | | |
| 2 | SEQ ID | TRB | CASSFSTCSANYGYTF | 3 | 0.60% |
| | NO: 1 | | | | |
| 3 | SEQ ID | TRB | CASSFSTCSANYGYTF | 2 | 0.40% |
| | NO: 1 | | | | |
| 4 | SEQ ID | TRB | CASFSTCSANYGYTF | 1 | 0.20% |
| | NO: 3 | | | | |
| 5 | SEQ ID | TRB | CASSFSTCSANYGYTF | 1 | 0.20% |
| | NO: 1 | | | | |
| 6 | SEQ ID | TRB | CASSFSTCSANYGYTF | 1 | 0.20% |
| | NO: 1 | | | | |
| 7 | SEQ ID | TRB | CASSFSTCSANYGYTF | 1 | 0.20% |
| | NO: 1 | | | | |
| 8 | SEQ ID | TRB | CASSFSTCSANYGYTF | 1 | 0.20% |
| | NO: 1 | | | | |
| 9 | SEQ ID | TRB | CASSFSTCSANYGYTF | 1 | 0.20% |
| | NO: 1 | | | | |
| 10 | SEQ ID | TRB | CASSFSTCSANYGYTF | 1 | 0.20% |
| | NO: 1 | | | | |

Collectively, the data demonstrate the efficiency of the methods described in Example 1 as a method to enrich target nucleic acids in situ, including target nucleic acids encoding for antibodies and immune cell receptors (e.g., B cell receptors, T cell receptors) and to enrich 5 for 5′ capture of target nucleic acids.

What is claimed is:

1. A method for determining locations of target nucleic acids in a biological sample, the method comprising:
(a) contacting the biological sample with a plurality of primers, wherein the plurality of primers comprises nucleic acid sequences that are substantially complementary to sequences in the target nucleic acids and a functional domain;
(b) hybridizing the plurality of primers to the target nucleic acids and extending one or more of the plurality of primers using the target nucleic acids as a template to generate one or more extension products;
(c) incorporating a polynucleotide sequence comprising at least three nucleotides to the 3′ end of the one or more extension products;
(d) hybridizing a second primer to the polynucleotide sequence comprising the at least three nucleotides of the one or more extension products of (c), wherein the second primer comprises a capture sequence;
(e) extending the one or more extension products using the second primer as a template, thereby incorporating a complement of the capture sequence into the one or more extension products;
(f) hybridizing the complement of the capture sequence of the one or more extension products of (e) to capture domains on an array, wherein the array comprises a plurality of capture probes, and wherein the plurality of capture probes collectively comprises spatial barcodes and the capture domains; and
(g) determining (i) the sequences of the spatial barcodes, or complements thereof, and (ii) all or a portion of the sequence of the target nucleic acids, or complements thereof, and using the determined sequences of (i) and (ii) to determine the locations of the target nucleic acids in the biological sample.

2. The method of claim 1, wherein the biological sample is disposed on the array comprising the plurality of capture probes.

3. The method of claim 1, wherein the biological sample is disposed on a first substrate, and wherein the array comprising the plurality of capture probes is disposed on a second substrate, and wherein the method further comprises aligning the first substrate with the second substrate, such that at least a portion of the biological sample is aligned with at least a portion of the array.

4. The method of claim 1, wherein the hybridizing in step (f) comprises passive migration or active migration, and optionally, wherein the active migration comprises electrophoresis.

5. The method of claim 1, wherein two or more primers in the plurality of primers hybridize to different sequences in a target nucleic acid of the target nucleic acids, wherein the method further comprises generating two or more extension products from two or more primers, optionally wherein the two or more extension products comprise different sequence lengths.

6. The method of claim 1, wherein a primer in the plurality of primers comprises a random sequence, preferably wherein the random sequence is a random hexamer or a random decamer.

7. The method of claim 1, wherein a primer in the plurality of primers comprises a homopolymer sequence, optionally wherein the homopolymer sequence comprises a poly(T) sequence.

8. The method of claim 1, wherein a primer of the plurality of primers comprises a sequence substantially complementary to a sequence in a target nucleic acid of the target nucleic acids encoding:
a constant region of an immune cell receptor, optionally a B cell receptor or a T cell receptor;
V and J sequences of an immune cell receptor; or
V, D, and J sequences of an immune cell receptor.

9. The method of claim 1, wherein the target nucleic acids comprise RNA, optionally wherein the RNA is mRNA.

10. The method of claim 9, wherein the mRNA comprises a sequence encoding a T cell receptor or a fragment thereof, or a B cell receptor or a fragment thereof.

11. The method of claim 1, wherein the extending in step (b) comprises: (i) the use of a reverse transcriptase with strand displacing activity; (ii) the use of a reverse transcriptase and a helicase; or (iii) the use of a reverse transcriptase and a superhelicase.

12. The method of claim 11, wherein (ii) further comprises the use of one or more single-stranded DNA binding proteins, optionally wherein the one or more single-stranded DNA binding proteins comprises one or more of: Tth RecA, E. coli RecA, T4 gp32 and ET-SSB.

13. The method of claim 11, wherein the superhelicase in (iii) comprises Rep, PrcA, UvrB, RecBCD, or Tte-Uvrd, and wherein the superhelicase has strand displacement activity.

14. The method of claim 1, wherein incorporating of the polynucleotide sequence to the 3′ end of the one or more extension products in step (c) comprises the use of a terminal deoxynucleotidyl transferase or a reverse transcriptase.

15. The method of claim 1, wherein the method further comprises removing the target nucleic acids, before the complement of the capture sequence of the one or more extension products hybridizes to the plurality of capture domains of the capture probes on the array, with: (i) an RNase, optionally wherein the RNase is RNase H, or (ii) heat.

16. The method of claim 1, wherein the method further comprises a step of extending the 3′ end of the one or more extension products of step (e) using the capture probes as templates, thereby generating one or more extended capture products, and/or extending the capture probes using the one or more extension products of step (e) as templates, thereby generating one or more extended capture probes, optionally, wherein the one or more extended capture products are removed from the capture probes on the array.

17. The method of claim 16, wherein the determining in step (g) comprises sequencing the one or more extended capture products, or amplicons thereof, or the one or more extended capture probes, or amplicons thereof.

18. The method of claim 1, wherein the method further comprises:
fixing the biological sample, wherein fixing the biological sample comprises the use of a fixative selected from the group consisting of: ethanol, methanol, acetone, formaldehyde, paraformaldehyde-Triton, glutaraldehyde, and combinations thereof; and/or
staining the biological sample and/or imaging the biological sample, optionally wherein the staining comprises: (i) use of eosin and/or hematoxylin, or (ii) use of a detectable label selected from the group consisting of a radioisotope, a fluorophore, a chemiluminescent compound, a bioluminescent compound, or a combination thereof.

19. The method of claim 1, wherein the method further comprises permeabilizing the biological sample, wherein the permeabilizing comprises use of an endopeptidase, a protease, sodium dodecyl sulfate, polyethylene glycol tert-octylphenyl ether, polysorbate 80, polysorbate 20, N-lauroylsarcosine sodium salt solution, saponin, or a combination thereof.

20. The method of claim 1, wherein the capture probe further comprises one or more functional domains, a unique molecular identifier, a cleavage domain, or a combination thereof.

21. The method of claim 1, wherein the biological sample is a tissue section, optionally wherein the tissue section is a fresh frozen tissue section, a formalin-fixed paraffin-embedded tissue section, a paraformaldehyde-fixed tissue section, a methanol-fixed tissue section, or an acetone-fixed tissue section.


SEQUENCE LISTING (3 sequences)

SEQ ID NO: 1 - protein, 16 aa, Homo sapiens
CASSFSTCSANYGYTF

SEQ ID NO: 2 - protein, 17 aa, Homo sapiens
CAVSDLEPNSSASKIIF

SEQ ID NO: 3 - protein, 15 aa, Homo sapiens
CASFSTCSANYGYTF